(19) United States
(12) Patent Application Publication
(10) Pub. No.: US 2021/0002718 A1
(43) Pub. Date: Jan. 7, 2021

(54) QUANTITATIVE MICROBIAL COMMUNITY PROFILING USING MOLECULAR INVERSION PROBES WITH UNIQUE MOLECULAR IDENTIFIERS

(71) Applicant: Trace Genomics, Inc., Burlingame, CA (US)

(72) Inventors: Zachary ZWIRKO, Burlingame, CA (US); Wyatt HARTMAN, Burlingame, CA (US)

(73) Assignee: Trace Genomics, Inc., Burlingame, CA (US)

(21) Appl. No.: 16/918,093

(22) Filed: Jul. 1, 2020

Related U.S. Application Data
(60) Provisional application No. 62/869,231, filed on Jul. 1, 2019.

(51) Int. Cl. C12Q 1/6869; C12Q 1/6809; C12Q 1/683

(52) U.S. Cl. CPC C12Q 1/6869; C12Q 1/6809; C12Q 1/683; C12Q 2531/113; C12Q 2521/501; C12Q 2521/319; C12Q 2600/156

(57) ABSTRACT

The present disclosure relates to the profiling of microorganisms in an environmental sample. Specifically, the present disclosure relates to methods of using molecular inversion probes comprising unique molecular identifiers to profile microorganisms in an environmental sample. In addition, the present disclosure relates to compositions of molecular inversion probes comprising unique molecular identifiers to profile microorganisms in an environmental sample.

### CROSS-REFERENCE TO RELATED APPLICATION

This application claims the benefit of U.S. Provisional Application 62/869,231, filed Jul. 1, 2019, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 794802000100SEQLIST.TXT, date recorded: Jun. 29, 2020, size: 9 KB).

### FIELD OF THE INVENTION

The present invention relates to specialized molecular inversion probes (MIPs), methods of using the same, and methods of profiling microorganisms in an environmental sample.

### BACKGROUND

Current methods for profiling microbial communities in environmental samples by nucleic acid sequencing rely on random fragmentation of microbial community DNA and subsequent whole genome library construction followed by shotgun sequencing. The resulting sequencing reads are then aligned to a collection of reference sequences for identification and enumeration. These methods, however, suffer from significant limitations, including ambiguous alignment of reads to multiple genomes and lack of annotation in the associated reference sequences, which lead to approximately 90% of sample data obtained from shotgun sequencing not being assigned or enumerated. Furthermore, the “hit rate”, which refers to the percentage of reads that are successfully and unambiguously mapped to a reference sequence, varies significantly across samples, so it is difficult to predict how many reads are needed to achieve a minimal hit threshold. This often results in sample reruns, which greatly increase turnaround time and costs.

In addition, current amplification-based metagenomic methods do not distinguish polymerase chain reaction (PCR) duplicate molecules from original progenitor molecules, impeding accurate quantification of microbial community members. Furthermore, PCR efficiency is known to be influenced by a number of factors including GC-content, sequence length, potential for formation of secondary structures, and template concentration. Inaccurate estimates of microbial abundance may be especially problematic for risk assessment of soil pathogens in a soil sample, as the likelihood of disease is directly proportional to overall microbial concentration. Furthermore, PCR is known to introduce errors, which may introduce false-positive calls and confound enumeration of low-level subpopulations that may be biochemically relevant.

Accordingly, there is a need in the art for a targeted sequencing method for profiling microbial communities, where well-annotated targets are identified, enriched in sequencing libraries, accurately quantified, and sequencing and/or amplification errors are corrected.

### BRIEF SUMMARY

The invention provides specialized molecular inversion probes (MIPs) and methods of using the same. The methods provided herein enable profiling microorganisms in an environmental sample by sequencing using these MIPs. The methods of profiling microorganisms described herein provide several advantages over methods existing in the art. For example, the methods described herein enable the accurate quantification of microbial abundance and/or genomic locus content within an environmental sample, while other methods are subject to errors due to duplications and/or bias during amplification, errors during sequencing and/or amplification, and suffer from significant limitations, including ambiguous alignment of reads to multiple genomes, and lack of annotation in the associated reference sequences. The methods provided herein also permit correction of sequencing and/or amplification errors and identification of genomic locus sequence variants in an environmental sample.

In one aspect, the invention provides a method for profiling of microorganisms in an environmental sample, wherein the method includes the steps of:
a) extracting DNA from the environmental sample;
b) denaturing the extracted DNA;
c) incubating the denatured DNA with a molecular inversion probe (MIP) under conditions that allow hybridization,
wherein the MIP is composed of
   (i) in the 3′ to 5′ direction,
   a first target locus primer, wherein the first primer includes a nucleotide sequence complementary to a first sequence in a target locus,
   a universal backbone sequence including a first sequencing primer binding site and a second sequencing primer binding site, and
   a second target locus primer, wherein the second primer includes a nucleotide sequence complementary to a second, non-overlapping sequence in the target locus and
   (ii) a first unique molecular identifier (UMI);
wherein the backbone sequence has low sequence homology to DNA in the environmental sample and has minimal ability to form secondary structures,
thereby generating a sample containing denatured DNA-MIP complexes;
d) after hybridization, performing an extension and ligation reaction that involves incubating the sample containing denatured DNA-MIP complexes with nucleotides, 5′ exo-polymerase lacking strand displacement activity, and a thermostable ligase capable of ligating splinted substrates under conditions that allow extension of the 3′ end of the MIP and ligation to the 5′ end of the MIP;
e) after extension and ligation, incubating the sample containing denatured DNA-MIP complexes with a 3′ to 5′ single strand exonuclease and a 3′ to 5′ double strand exonuclease under conditions sufficient to degrade linear substrates, thereby generating a sample containing circular DNA templates;
f) removing the 3′ to 5′ single strand exonuclease and the 3′ to 5′ double strand exonuclease from the sample containing circular nucleic acid templates;
g) amplifying the circular DNA templates, thereby generating linear DNA containing the sequence of the MIP from 5′ end of the first primer binding site to the 3′ end of the second primer binding site; and
h) sequencing the linear DNA, thereby generating a plurality of sequencing reads containing the sequence of the linear DNA, thereby profiling the microorganisms in the environmental sample.

In some embodiments, the first UMI is between the first target locus primer and the first sequencing primer binding site. In certain embodiments, the MIP contains a second UMI. In certain embodiments, the second UMI is between the second target locus primer and the second sequencing primer binding site. In certain embodiments, the first UMI and the second UMI each are composed of between 5 and 20 bases.

In some embodiments, which may be combined with any of the preceding embodiments, the first target locus primer and the second target locus primer contain at least one degenerate nucleotide base at the 3′ end and/or the 5′ end.

In some embodiments, which may be combined with any of the preceding embodiments, the denatured DNA is incubated with a second MIP containing a first and a second target locus primer complementary to sequences in a second target locus.

In some embodiments, which may be combined with any of the preceding embodiments, the 5′ exo-polymerase lacking strand displacement activity is Stoffel fragment, TaqIT, Klenow large fragment, or Phusion polymerase.

In some embodiments, which may be combined with any of the preceding embodiments, the thermostable ligase capable of ligating splinted substrates is Taq ligase, T4 DNA ligase, or Ampligase.

In some embodiments, which may be combined with any of the preceding embodiments, the 3′ to 5′ single strand exonuclease is exonuclease I. In some embodiments, which may be combined with any of the preceding embodiments, the 3′ to 5′ double strand exonuclease is exonuclease III or Kamchatka crab nuclease. In certain embodiments, which may be combined with any of the preceding embodiments, the 3′ to 5′ single strand exonuclease and the 3′ to 5′ double strand exonuclease are removed by heat inactivation and/or purification.

In certain embodiments, which may be combined with any of the preceding embodiments, the step of amplifying includes a polymerase chain reaction (PCR) including a PCR reaction mix, wherein the PCR reaction mix contains a high-fidelity proof-reading polymerase and sequencing primers. In some embodiments, the sequencing primers contain a sequence complementary to the first or second sequencing primer binding sites, and a P5 or a P7 sequence. In certain embodiments, the sequencing primers also contain a sample index.

In some embodiments, which may be combined with any of the preceding embodiments, the sequencing involves sequencing with massively parallel sequencing using reversible chain termination. In certain embodiments, which may be combined with any of the preceding embodiments, the sequencing reads are paired-end reads.

In some embodiments, the method also includes the step of grouping sequencing reads if they contain the same sample index sequence, thereby generating bins containing sequencing reads from the same sample.

In some embodiments, which may be combined with any of the preceding embodiments, the method also includes the step of grouping the sequencing reads from the same sample if they contain the same UMI sequence, thereby generating bins containing sequencing reads from the same sample and with the same UMI sequence, thereby quantifying the number of unique target loci in the sample. In certain embodiments, the method also includes the step of analyzing the sequencing reads from the same sample and with the same UMI sequence to determine whether the sequencing reads have the same or a different nucleotide at each position.

In certain embodiments, which may be combined with any of the preceding embodiments, the method also includes the step of aligning the sequencing reads to a collection of functional and phylogenetic reference sequences, thereby identifying the microorganisms in the environmental sample. In certain embodiments, the method also includes the step of analyzing the sequencing reads from the same sample and target locus to determine whether the sequencing reads have the same or a different nucleotide at each position relative to a functional and phylogenetic reference sequence.

In certain embodiments, the method also includes the step of determining microbial abundance in the environmental sample based on the number of unique target loci in the environmental sample. In certain embodiments, the method also includes the step of determining chemical availability and/or Transformation Process Rates in the environmental sample based on the number of unique target loci and/or the microorganisms identified in the environmental sample.

In certain embodiments, which may be combined with any of the preceding embodiments, a known amount of a spike-in is added to the environmental sample prior to the step of extracting DNA from the environmental sample, wherein the spike-in contains bacterial cells, fungal cells, or viral particles, and combinations thereof. In certain embodiments, which may be combined with any of the preceding embodiments, a known amount of a spike-in is added to the extracted DNA, wherein the spike-in contains DNA constructs, synthetic DNA, or DNA fragments, and any combinations thereof.

In certain embodiments, which may be combined with any of the preceding embodiments, the target locus is a taxonomic marker, including a 16S ribosomal RNA, an 18S ribosomal RNA, an internal transcribed spacer (ITS) region, a microbial sequence that identifies a species and/or strain, or a target locus that distinguishes a pathogenic microorganism from a non-pathogenic and/or beneficial microorganism.

In certain embodiments, which may be combined with any of the preceding embodiments, the target locus is a gene associated with a biological pathway, including

cycling or transformation of compounds containing nitrogen, nitrogen fixation, ammonia oxidation, nitrification, denitrification, organic nitrogen mineralization, mineral nitrogen immobilization, organic nitrogen immobilization,
cycling or transformation of compounds containing phosphorous mineral phosphorous solubilization, hydrolysis of organic phosphorous compounds, hydrolysis of inorganic phosphorous polymers, immobilization of phosphorous,
cycling or transformation of compounds containing carbon, uptake or degradation of sugars, uptake or degradation of oligosaccharides, uptake or degradation of polysaccharides, uptake or degradation of structural polymers,
uptake or degradation of cellulose, uptake or degradation of hemicellulose, uptake or degradation of lignocellulose, uptake or degradation of lignin,
uptake or degradation of aliphatic compounds, uptake or degradation of alkane compounds, uptake or degradation of aromatic compounds,
metabolic pathways for aerobic respiration, metabolic pathways for anaerobic respiration, aerobic cytochrome oxidation, microaerobic cytochrome oxidation, anaerobic respiration utilizing nitrate, iron, manganese, sulfate, acetate, or CO₂ as terminal electron acceptors, or anaerobic cytochrome oxidation, and any combinations thereof.

In certain embodiments, which may be combined with any of the preceding embodiments, the target locus is a gene associated with a processes, including agricultural processes, plant growth, plant disease, cycling of micronutrients, cycling of potassium, cycling of zinc, cycling of calcium, plant growth promotion, production of indole-3-acetic acid (IAA), production of siderophores, production of 1-amino-cyclopropane-1-carboxylate (ACC) deaminase, production of hydrogen cyanate, nutrition, N-fixation, P solubilization, disease suppression in the soil, or antibiotic resistance, and any combinations thereof.

In some embodiments, which may be combined with any of the preceding embodiments, the environmental sample contains soil. In certain embodiments, which may be combined with any of the preceding embodiments, the environmental sample contains bacterial cells, fungal cells, nematodes, and/or virus particles.

In another aspect, the invention provides compositions for profiling of microorganisms in an environmental sample including, a molecular inversion probe (MIP), wherein the MIP includes
(i) in the 3′ to 5′ direction,
a first target locus primer, wherein the first primer contains a nucleotide sequence complementary to a first sequence in a target locus,
a universal backbone sequence containing a first sequencing primer binding site and a second sequencing primer binding site, and
a second target locus primer, wherein the second primer contains a nucleotide sequence complementary to a second, non-overlapping sequence in the target locus and
(ii) a first unique molecular identifier (UMI);
wherein the backbone sequence has low sequence homology to DNA in the environmental sample and has minimal ability to form secondary structures.

In certain embodiments, the first UMI is between the first target locus primer and the first sequencing primer binding site. In some embodiments, the MIP also contains a second UMI. In certain embodiments, the second UMI is between the second target locus primer and the second sequencing primer binding site. In certain embodiments, the first UMI and the second UMI each are composed of between 5 and 20 bases.

In certain embodiments, which may be combined with any of the embodiments of this aspect, the first target locus primer and the second target locus primer contain at least one degenerate nucleotide base at the 3′ end and/or the 5′ end.

In certain embodiments, which may be combined with any of the embodiments of this aspect, the composition also contains a second MIP, wherein the second MIP contains a first and a second target locus primer complementary to sequences in a second target locus.

In some embodiments, which may be combined with any of the embodiments of this aspect, the target locus is a taxonomic maker, including a 16S ribosomal RNA, an 18S ribosomal RNA, an internal transcribed spacer (ITS) region, a microbial sequence that identifies a species and/or strain, or a target locus that distinguishes a pathogenic microorganism from a non-pathogenic and/or beneficial microorganism.

In some embodiments, which may be combined with any of the embodiments of this aspect, the target locus is a gene associated with a biological pathway, including

cycling or transformation of compounds containing nitrogen, nitrogen fixation, ammonia oxidation, nitrification, denitrification, organic nitrogen mineralization, mineral nitrogen immobilization, organic nitrogen immobilization,
cycling or transformation of compounds containing phosphorous mineral phosphorous solubilization, hydrolysis of organic phosphorous compounds, hydrolysis of inorganic phosphorous polymers, immobilization of phosphorous,
cycling or transformation of compounds containing carbon, uptake or degradation of sugars, uptake or degradation of oligosaccharides, uptake or degradation of polysaccharides, uptake or degradation of structural polymers,
uptake or degradation of cellulose, uptake or degradation of hemicellulose, uptake or degradation of lignocellulose, uptake or degradation of lignin,
uptake or degradation of aliphatic compounds, uptake or degradation of alkane compounds, uptake or degradation of aromatic compounds,
metabolic pathways for aerobic respiration, metabolic pathways for anaerobic respiration, aerobic cytochrome oxidation, microaerobic cytochrome oxidation, anaerobic respiration utilizing nitrate, iron, manganese, sulfate, acetate, or CO₂ as terminal electron acceptors, or anaerobic cytochrome oxidation, and any combinations thereof.

In some embodiments, which may be combined with any of the embodiments of this aspect, the target locus is a gene associated with a process, including agricultural processes, plant growth, plant disease, cycling of micronutrients, cycling of potassium, cycling of zinc, cycling of calcium, plant growth promotion, production of indole-3-acetic acid (IAA), production of siderophores, production of 1-amino-cyclopropane-1-carboxylate (ACC) deaminase, production of hydrogen cyanate, nutrition, N-fixation, P solubilization, disease suppression in the soil, or antibiotic resistance, and any combinations thereof.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts a design for exemplary molecular inversion probes (MIPs) for use in profiling of microorganisms in environmental samples. The MIP includes two target locus primers, “A” and “B”, also termed “extension arm” and “ligation arm”, respectively. The sequences of the target locus primers are complementary to non-overlapping sequences that flank a target locus. The target locus primers are connected by a universal backbone sequence. Adjacent to each target locus primer is a unique molecular identifier (“UMI-1” and “UMI-2”) sequence consisting of between 5 and 20 degenerate nucleotides. The universal backbone sequence also contains two sequencing primer-binding sites (“SP1” and “SP2”). The 3′ and 5′ ends of the MIP are indicated.

FIGS. 2A-2D depict the hybridization, extension and ligation, and exonuclease degradation steps of an exemplary method for profiling microorganisms in environmental samples provided herein. As shown in FIG. 2A, following denaturation of sample nucleic acids in the presence of MIPs, the samples are cooled to a temperature that allows hybridization of the target locus primers on the MIP to their complementary sequences flanking the target locus in the sample nucleic acids, thereby circularizing the MIP. Following hybridization, as shown in FIG. 2B, a 5′exo-polymerase lacking strand displacement activity synthesizes a sequence complementary to the target sequence starting at the 3′ end of target locus primer “A” until reaching target locus primer “B” at the 5′ end of the MIP. A thermostable ligase capable of splinted ligation then ligates the newly synthesized 3′ end of the target sequence to the 5′-phosphorylated end of the MIP, creating an uninterrupted circular molecule (FIG. 2C). The samples are then incubated with a 3′ to 5′ single strand exonuclease and a 3′ to 5′ double strand exonuclease to degrade genomic DNA, cDNA, and unused MIPs (FIG. 2D). The exonucleases are then removed by heat-inactivation or purification of the nucleic acid samples.

FIGS. 3A-3B depict the amplification and preparation of samples for sequencing steps of an exemplary method for profiling microorganisms in environmental samples provided herein. As shown in FIG. 3A, the circularized MIPs are amplified by PCR using sequencing primers that bind to the SP1 and SP2 sites on the MIP. The sequencing primers also contain a sequencing adapter “primer tail” (“P5” and “P7”) and sample index sequences (not depicted). The PCR reactions generate linear double stranded DNA products that contain the sequencing adapters, UMI sequences (UMI-1 and UMI-2), the target locus sequence, and the sample index (FIG. 3B).

FIGS. 4A-4C depict the sequencing, demultiplexing, enumeration and error correction steps of the method for profiling microorganisms in environmental samples provided in Example 2. As shown in FIG. 4A, sequencing of the samples is performed with massively parallel sequencing using reversible chain termination. The resulting sequencing reads are demultiplexed by grouping the reads according the sample index sequences, such that all reads carrying the same index sequence are grouped (“Demultiplexing by Sample Index”). Each group of reads corresponding to a single sample index sequence is then demultiplexed by grouping the reads according to the UMI sequence, such that all reads carrying the same UMI sequence are grouped (“Enumeration by UMI Sequence”). Alignment of the reads to reference nucleic acid collections can be performed prior to or after the demultiplexing and enumeration steps. As depicted in FIG. 4B, the number of unique progenitor target locus molecules present in the nucleic acid sample prior to amplification is quantified by collapsing sequencing reads with the same UMI sequence and the same target sequence. FIG. 4C depicts sequencing data showing two counts of the 16S rRNA gene sequence with UMI 1, three counts with UMI 2, and three counts with UMI 3. The two counts of the sequence with UMI 1 are duplicates, as are the three counts with UMI 2 and the three counts with UMI3, therefore there were three original progenitor molecules corresponding to the 16S rRNA gene in the sample. As shown in FIG. 4D, PCR and/or sequencing errors are corrected by forming a molecular consensus across reads carrying the same UMI and target sequence. “X” indicates a sequence variant or mutation within the target locus sequence.

FIGS. 5A-5E depict the quantification of microbial abundances in an environmental sample as described in Example 3. FIG. 5A depicts sequencing data showing two reads with UMI 1 mapping to a 16S rRNA sequence belonging to the Sinorhizobium genus of nitrogen-fixing bacteria, and three reads with each one of UMI 2 and UMI 3 mapping to a 16S rRNA sequence belonging to the Methylosinus genus of methanotrophic bacteria. As shown in FIG. 5B, the respective relative abundance of the Sinorhizobium and Methylosinus genera in the sample is 1:2. FIG. 5C depicts sequencing data showing two reads with UMI 4 mapping to an ITS spacer region of the fungal pathogen Fusarium, and three reads with UMI 5 mapping to an ITS spacer region of the fungal symbiont Glomus. As shown in FIG. 5D, the relative abundance of Fusarium to Glomus in the sample is 1:1. FIG. 5E depicts the use of a spike-in to determine the absolute abundances of target loci. A known amount of the 16S rRNA locus standard is spiked into a DNA extract from a soil sample containing bacterial cells of the Sinorhizobium and Methylosinus taxa. As shown in FIG. 5E, following sequencing, the absolute abundances of Sinorhizobium and Methylosinus are determined based on the measured abundance and known amount of the 16S rRNA locus standard. The vertical black line represents the known amount of 16S rRNA locus standard.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### Definitions

The following definitions are provided to facilitate understanding of certain terms used frequently herein and are not meant to limit the scope of the present disclosure.

As used herein, the terms “complementary” and “complementarity” refer to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared with appropriate nucleotide insertions or deletions, pair with at least about 80% of the nucleotides of the other strand, at least about 90% to 95%, or at least about 98 to 100%. Alternatively, complementarity exists when an RNA or DNA strand will hybridize under selective hybridization conditions to its complement. Typically, selective hybridization will occur when there is at least about 65% complementarity over a stretch of at least 14 to 25 nucleotides, at least about 75%, or at least about 90% complementarity. See, M. Kanehisa Nucleic Acids Res. 12:203 (1984), incorporated herein by reference.

As used herein, the term “read” refers to sequence data from a portion of a nucleic acid sample. Typically, though not necessarily, a read represents a short sequence of contiguous base pairs in the sample. The read may be represented symbolically by the base pair sequence of the sample portion. It may be stored in a memory device and processed as appropriate to determine whether it matches a reference sequence or meets other criteria. A read may be obtained directly from a sequencing apparatus or indirectly from stored sequence information concerning the sample. In some embodiments, sequencing is carried out using single-end or paired-end reads. Sequencing reads are of sufficient length to identify a larger sequence or region, e.g., that can be aligned and specifically assigned to a chromosome or genomic region or gene. For example, a read may be at least about 75 bp, at least about 100 bp, at least about 125 bp, at least about 150 bp, at least about 175 bp, at least about 200 bp, at least about 225 bp, at least about 250 bp, at least about 275 bp, at least about 300 bp, or at least about 325 bp in length.

As used herein, the terms “index”, “barcode”, “sample index”, and “index sequence” are used interchangeably unless specified otherwise. The terms refer to a sequence of nucleotides, usually oligonucleotides, that can be used to identify a sequence of interest. The index sequence may be exogenously incorporated into the sequence of interest by ligation, extension, or other methods known in the art. The index sequence may also be endogenous to the sequence of interest, e.g., a fragment in the sequence of interest itself may be used as an index. For implementations of index sequences, see, Kinde, et al. (2011), Proceedings of the National Academy of Sciences, 108, 9530.

As used herein, the terms “align”, “aligned,” “alignment,” or “aligning” refer to the process of comparing a sequencing read to a reference sequence and thereby determining whether the reference sequence contains the read sequence. Aligned reads are one or more sequences that are identified as a match in terms of the order of their nucleic acid molecules to a known sequence from a reference genome. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). In some cases, an alignment additionally indicates a location in the reference sequence where the read maps to.

The terms “mapping”, “mapped” and “map” as used herein refer to specifically assigning a sequence read to a larger sequence, e.g., a reference genome, by alignment.

As used herein, the term “reference genome” or “reference sequence” refers to any particular known genome sequence, whether partial or complete, of any organism which may be used to align and/or map identified sequences from a sample. For example, reference sequences include genomes of bacterial, fungal, and viral species, as well as chromosomes, extra-chromosomal elements (e.g., plasmids), sub-chromosomal regions (such as strands) thereof. In some examples, references include genomes, cDNA sequences, chromosomes, extra-chromosomal elements, and sub-chromosomal regions of any species.

As used herein, the term “spike-in” refers to a molecule(s) (e.g., nucleic acid molecule), cell, or organism (e.g., a microbial organism) that is added to a sample in a known amount and that serves as a control for the sample. In some embodiments, the spike-in is a molecule, cell, or organism. In some embodiments, the spike-in is a molecule, cell, or organism that has a detectable tag, barcode, or sequence that facilitates the identification of the spike-in in the sample. In some embodiments, a spike-in is a synthetic nucleic acid molecule (e.g., a synthetic DNA or RNA oligonucleotide). In some embodiments, a spike-in is a cell or organism (e.g., a bacterial species, phytoplasma species, viral species, viroid species, rickettsia species, fungal species, helminth species, protozoan, parasite species, or pest species).

As used herein, the terms “nucleic acid” and “polynucleotide” interchangeably refer to deoxyribonucleotide (DNA) or ribonucleotide (RNA) and polymers thereof in either single- or double-stranded form. The term encompasses nucleic acids containing known nucleotide analogs or modified backbone residues or linkages, which are synthetic, naturally occurring, and nonnaturally occurring, which have similar binding properties as the reference nucleic acid, and which are metabolized in a manner similar to the reference nucleotides. Examples of such analogs include, without limitation, phosphorothioates, phosphoramidates, methyl phosphonates chiral-methyl phosphonates, 2-O-methyl ribonucleotides, and peptide nucleic acids (PNAs). In certain applications, the nucleic acid can be a polymer that includes multiple monomer types, e.g., both RNA and DNA subunits.

As used herein, the term “target” refers to a molecule or organism whose detection and/or quantification is intended. In some embodiments, a target is a nucleic acid sequence (e.g., an extracted nucleic acid sequence in a population of nucleic acid sequences). In some embodiments, a target is a nucleic acid sequence of a bacterial species, viral species, fungal species, nematode species, parasite species, or pest species.

As used herein, the terms “about” or “approximately” mean a range of values, including the specified value, which a person of ordinary skill in the art would consider reasonably similar to the specified value. In some embodiments, the term “about” or “approximately” means with a standard deviation using measurements generally acceptable in the art. In some embodiments, the term “about” or “approximately” means a range extending to ±10% of the specified value. In some embodiments, the term “about” or “approximately” means the specified value.

As used herein, the term “comprise,” and variations thereof such as “comprises” and “comprising”, when preceding the recitation of a step or an element, are intended to mean that the addition of further steps or elements is optional and not excluded. Any methods, devices and materials similar or equivalent to those described herein can be used in the practice of this invention.

As used herein, the term “low sequence homology” refers to a given nucleic acid sequence having insufficient complementarity to cause non-specific intra-molecular or inter-molecular binding at hybridization temperature. For example, in some embodiments, the backbone sequence of a MIP has less than 50, less than 25, less than 20, less than 10, or less than 5 consecutive nucleotides that are 100%, at least 90%, at least 80%, at least 70%, or at least 65% complementary to any nucleic acids present in an environmental sample.

### Overview

The present disclosure provides specialized molecular inversion probes (MIPs), i.e., MIP-UMIs, for profiling microorganisms in an environmental sample. The present disclosure also provides methods for profiling microorganisms in an environmental sample by sequencing using these MIPs.

The methods for profiling microorganisms described herein address the limitations of current methods by targeting specific genomic loci of interest for sequencing using the MIPs provided herein. In contrast, current methods for profiling microorganisms by sequencing, such as shotgun sequencing, suffer from significant limitations including ambiguous alignment of reads to multiple genomes and lack of annotation in the associated reference sequences, which lead to approximately 90% of sample data obtained from current sequencing methods not being assigned or enumerated. Furthermore, the “hit rate” varies significantly across samples using current methods, so it is difficult to predict how many reads are needed to achieve a minimal hit threshold. This often results in sample reruns, which greatly increase turnaround time and costs.

The methods for profiling microorganisms described herein use MIPs with unique molecular identifiers (UMIs) to accurately quantify microbial abundance and/or genomic locus content within an environmental sample. The methods provided herein use the diversity of these specialized MIPs to quantify unique progenitor molecules for each target locus. In contrast, current methods (e.g., read counting alone) do not distinguish original progenitor molecules from duplicate molecules arising from amplification reactions and do not account for biases in amplification and/or sequencing, impeding accurate quantification of microbial community members.

The methods for profiling microorganisms using the MIPs described herein also enable the correction of amplification and sequencing errors, as well as identification of sequence variants in environmental samples by building a molecular sequence consensus using UMI sequences. In contrast, current methods do not distinguish sequencing and amplification errors from sequence variants, and may lead to false positive calls and confound enumeration of low-level subpopulations that may be biochemically relevant.

Furthermore, while current amplification-based metagenomic methods typically infer biochemical activity through the indirect association of phylogenetic information with putative functions of associated taxa, the methods of the present disclosure allow direct interrogation of both phylogenetic and functional markers in a single assay. This is particularly beneficial for samples where large fractions of the microbial community lack prior annotation, as the methods of the present invention provide insights into both the phylogeny and function of previously uncharacterized microbes. Thus, the methods disclosed herein are useful for iteratively expanding databases of metagenomic reference sequences, thereby allowing continual improvement of phylogenetic resolution.

### Compositions of the Disclosure

The specialized molecular inversion probes (MIPs) provided herein include first and second target locus primers with nucleotide sequences complementary to non-overlapping sequences on a target locus, a universal backbone sequence with first and second sequencing primer binding sites, low sequence homology to DNA in the environmental sample and minimal ability to form secondary structures, and at least one unique molecular identifier (UMI);

### Universal Backbone

In some embodiments, the universal backbone sequence has a length of between 20 and 1,000 base pairs (e.g., 20, 21, 22, 23, 24, 25, 26, 27, 28, 29, 30, 31, 32, 33, 34, 35, 36, 37, 38, 39, or 40 base pairs). In some embodiments, the universal backbone sequence has a melting temperature of between about 45° C. and about 80° C. (e.g., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., or about 65° C.). In some embodiments, the universal backbone sequence has a GC content between about 30% and about 80% (e.g., approximately about 30%, about 35%, about 40%, about 45%, about 50%, about 55%, about 60%, about 65%, about 70%, about 75%, or about 80%, or any range between about 30% and about 80%, such as 30-70%). In certain embodiments, the universal backbone sequence includes at least one primer binding site, e.g., one, two, or more sequencing primer binding sites.

In some embodiments, the universal backbone sequence has minimal ability to form secondary structures (i.e., insufficient complementarity to cause non-specific intra-molecular or inter-molecular binding at hybridization temperature). In certain embodiments, the universal backbone sequence has less than 50, less than 25, less than 20, less than 10, or less than 5 consecutive nucleotides that are 100%, at least 90%, at least 80%, at least 70%, or at least 65% complementary to any other consecutive nucleotides on the universal backbone sequence.

In some embodiments, the universal backbone sequence has low sequence homology to any DNA present in the sample (i.e., insufficient complementarity to cause non-specific inter-molecular binding at hybridization temperature). In some embodiments, the universal backbone sequence has less than 50, less than 25, less than 20, less than 10, or less than 5 consecutive nucleotides that are 100%, at least 90%, at least 80%, at least 70%, or at least 65% complementary to any DNA present in the sample.

A non-limiting example of a universal backbone sequence (SEQ ID NO: 1) is provided in Table 1.

### Unique Molecular Identifiers (UMI)

In some embodiments, a MIP contains one UMI. In certain embodiments, a MIP contains one UMI between a first target locus primer and a first primer binding site. In certain embodiments, a MIP contains two UMIs. In certain embodiments, a MIP contains a first UMI between a first target locus primer and a first primer binding site, and a second UMI between a second target locus primer and a second primer binding site. In some embodiments, a MIP contains a first UMI and a second UMI between a first target locus primer a first primer binding site.

The MIPs provided herein include one or more unique molecular identifier (UMI). In some embodiments, UMIs include a degenerate or semi-degenerate nucleotide sequence. In some embodiments, UMIs include a completely random and degenerate nucleotide sequence, wherein each sequence position may be any nucleotide (i.e., each position is not limited, and may an adenine (A), cytosine (C), guanine (G), thymine (T), or uracil (UC), or any other natural or non-natural DNA or RNA nucleotide or nucleotide-like substance or analog with base-pairing properties, including, but not limited to xanthosine, inosine, hypoxanthine, xanthine, 7-methylguanine, 7-methylguanosine, 5,6-dihydrouracil, 5-methylcytosine, dihydouridine, isocytosine, isoguanine, deoxynucleosides, nucleosides, peptide nucleic acids, locked nucleic acids, glycol nucleic acids and threose nucleic acids). In other embodiments, UMIs include a semi-degenerate nucleotide sequence wherein not all sequence positions are completely random and degenerate.

UMIs may be of any suitable length to produce a sufficiently large number of unique UMIs. In some embodiments, UMIs may be between 5 and 20 nucleotides in length. Therefore, each UMI may be approximately 5, 6, 7, 8, 9, 10, 11, 12, 13, 14, 15, 16, 17, 18, 19 or 20 nucleotides in length. In one embodiment, a UMI is a nucleotide sequence 5 nucleotides in length. In another embodiment, a UMI is a nucleotide sequence 10 nucleotides in length. In another embodiment, a UMI is a nucleotide sequence 20 nucleotides in length.

In some embodiments, the degenerate or semi-degenerate UMI sequences may be generated by a polymerase-mediated method. In certain embodiments, UMIs may be synthesized using 4-fold degenerate nucleotide approach (i.e. all four nucleotide precursors are added into reactions at about equimolar concentrations) or 3-fold degenerate nucleotide approach (e.g., A, T and C nucleotide precursors are added into reaction at about equimolar concentrations). In some embodiments, UMIs may be generated by preparing and annealing a library of individual oligonucleotides of known sequence. Alternatively, degenerate or semi-degenerate UMI sequences may be a randomly or non-randomly fragmented nucleic acids from any alternative source of nucleic acids that differs from the nucleic acids in the sample. In some embodiments, the alternative source of nucleic acids is a genome or plasmid derived from bacteria, an organism other than those found in the sample, or a combination of such alternative organisms or sources. The random or non-random fragmented DNA may be introduced into MIPs for use as UMIs. This may be accomplished through enzymatic ligation or any other method known in the art.

In some embodiments, UMI sequences are generated through chemical synthesis using reversible chain termination (e.g., Integrated DNA Technologies). During synthesis of oligonucleotides by reversible chain termination the first nucleotide in a given sequence is coupled to a crushed porous glass membrane with the 3′ end chemically blocked. That nucleotide is then deblocked and a solution containing the next base in the sequence is added, again using nucleotides that are chemically blocked at the 3′end to ensure only a single base is added. That base is then deblocked and the next desired base is added, and the process is then repeated until the desired oligo sequence is complete. To generate UMI sequences using reversible chain termination, all four nucleotides are added at the same time. Trillions of molecules are coupled to the glass membrane, so each molecule is coupled to a random base.

### Target Locus Primers

Target locus primers are polynucleotide sequences that hybridize to a nucleic acid sequence of a target locus and serve as a point of initiation of nucleic acid synthesis.

Target locus primers can be of a variety of lengths. In some embodiments, a primer is less than 100 nucleotides in length, for example 10-80 nucleotides in length. The length and sequences of primers for use in PCR can be designed based on principles known to those of skill in the art. See, e.g., PCR Protocols: A Guide to Methods and Applications, Innis et al., eds, 1990.

In some embodiments, target locus primers hybridize only to a perfect sequence match in the target locus. This can be accomplished, as known in the art, e.g., by adjusting the target locus primer length, GC percentage, hybridization conditions, melting temperature, and/or the like.

In some embodiments, the sequence of the target locus primers has 100% sequence complementarity to the target locus sequence. In some embodiments, target locus primers are mismatched at one or more nucleotides relative to the target locus sequence. In some embodiments, target locus primers include one or more random degenerate bases at the 3′ end. In some embodiments, target locus primers include one or more random degenerate bases at the 5′ end. In some embodiments, target locus primers include one or more random degenerate bases at the 3′ end and at the 5′ end. In some embodiments, target locus primers include 1, 2, 3, 4, 5, 6, 7, 8, 9, 10 or more random degenerate bases at the 3′ end and/or 1, 2, 3, 4, 5, 6, 7, 8, 9, 10 or more random degenerate bases at the 5′ end. In some embodiments, target locus primers include 5 random degenerate bases at the 3′ end or 5 random degenerate bases at the 5′ end.

### Target Loci

In some embodiments, the target loci are nucleic acid sequences of a bacterial species, phytoplasma species, viral species, viroid species, rickettsia species, fungal species, helminth species, protozoan, parasite species, or pest species. In some embodiments, the target loci are nucleic acid sequences of a pathogen (e.g., a pathogen that is a bacterial species, phytoplasma species, viral species, viroid species, rickettsia species, fungal species, helminth species, protozoan, parasite species, or pest species). In some embodiments, the target loci are nucleic acid sequences from a plant pathogen. In some embodiments, the target loci are nucleic acid sequences of a mammalian organism (e.g., a human, primate, equine, bovine, ovine, porcine, rodent, feline, or canine organism).

In some embodiments, the target loci are one or more nucleic acid sequences from one or more of the species listed in the “Samples” section below.

In some embodiments, the target loci are one or more nucleic acid sequences from one or more genes that function in the cycling and/or transformation of compounds containing nitrogen. In some embodiments, the genes function in nitrogen fixation (e.g., MfDHK, MfH), ammonia oxidation (e.g., amoABC), nitrification, denitrification (e.g., nirK), organic nitrogen mineralization, mineral nitrogen immobilization, or organic nitrogen immobilization, and any combinations thereof.

In some embodiments, the target loci are taxonomic markers selected from the group consisting of a 16S ribosomal RNA, an 18S ribosomal RNA, an internal transcribed spacer (ITS) region, a microbial sequence that identifies a species and/or strain, and a target locus that distinguishes a pathogenic microorganism from a non-pathogenic and/or beneficial microorganism.

In some embodiments, the target loci are one or more nucleic acid sequences from one or more genes that function in the cycling and/or transformation of compounds containing phosphorous. In some embodiments, the genes function in mineral phosphorous solubilization, hydrolysis of organic phosphorous compounds, hydrolysis of inorganic phosphorous polymers, or immobilization of phosphorous, and any combinations thereof.

In some embodiments, the target loci are one or more nucleic acid sequences from one or more genes that function in the cycling and/or transformation of compounds containing carbon. In some embodiments the genes function in uptake or degradation of sugars, uptake or degradation of oligosaccharides, uptake or degradation of polysaccharides, or uptake or degradation of structural polymers, and any combinations thereof.

In some embodiments, the target loci are one or more nucleic acid sequences from one or more genes that function in uptake or degradation of cellulose, uptake or degradation of hemicellulose, uptake or degradation of lignocellulose, uptake or degradation of lignin, uptake or degradation of aliphatic compounds, uptake or degradation of alkane compounds, or uptake or degradation of aromatic compounds, and any combinations thereof.

In some embodiments, the target loci are one or more nucleic acid sequences from one or more genes that function in metabolic pathways. In some embodiments the genes function in metabolic pathways for aerobic respiration, metabolic pathways for anaerobic respiration, aerobic cytochrome oxidation, microaerobic cytochrome oxidation, anaerobic respiration utilizing nitrate, iron, manganese, sulfate, acetate, or carbon dioxide as terminal electron acceptors, or anaerobic cytochrome oxidation, and any combinations thereof.

In some embodiments, the target loci are one or more nucleic acid sequences from one or more genes that function in agricultural processes, plant growth, plant disease, cycling of micronutrients, cycling of potassium, cycling of zinc, cycling of calcium, plant growth promotion, production of indole-3-acetic acid (IAA), production of siderophores, production of 1-amino-cyclopropane-1-carboxylate (ACC) deaminase, production of hydrogen cyanate, nutrition, N-fixation, P solubilization, disease suppression in the soil, or antibiotic resistance, and any combinations thereof.

In some embodiments, the target locus may be one or more conserved regions of each species target. In some embodiments, the target locus may be one or more divergent or highly evolving regions of each species target. For example, in some embodiments MIPs that target one or more conserved regions, highly evolved regions, or a combination thereof in a subset of species (e.g., a subset of bacterial species, phytoplasma species, viral species, viroid species, rickettsia species, fungal species, helminth species, protozoan species, parasite species, and/or pest species) may be used.

In some embodiments, the target loci may be one or more conserved bacterial regions and/or one or more highly evolving bacterial regions. For example, in some embodiments, bacterial target loci may be one or more 16S rRNA sequences. In some embodiments, the target loci may be one or more 16S rRNA sequences from the Sinorhizobium genus of nitrogen-fixing bacteria, from the Rhizobium genus of nitrogen-fixing bacteria, and/or from the Methylosinus genus of methanotrophic bacteria. 16S rRNA is known to be a common housekeeping sequence in bacteria that has both highly conserved and variable regions. See, e.g., Isenbarger et al., Orig Life Evol Biosph, 2008, DOI: 10.1007/sl 1084-008-9148-z. In some embodiments, the target loci may be one or more 16S rRNA sequences in combination with one or more other gene sequences in bacteria (e.g., one or more housekeeping genes or pathogenicity genes). For example, in some embodiments, a combination of 16S rRNA and one or more sequences selected from patl, atpD, dnaK, gyrB, ppK, recA, rpoB, HSPI, HSP4, hrpZ, cfl, gapl, rpoD, pgi, kup, acnB, gltA, hrpF, fusA, gapA, lacF, lepA, ppsA, adk, gdhA, hrpB, fliC, egl, gmc, ugpB, pilT, trpB, phaC, mutL, rpoB, and trpB can be targeted for identifying a bacterial species target at the species and subspecies or strain level.

In some embodiments, the target loci may be one or more conserved fungal regions and/or one or more highly evolving fungal regions. For example, in some embodiments, fungal target loci may be one or more 18S rRNA sequences. For example, in some embodiments, fungal target loci may be one or more fungal internal transcribed spacer (ITS) rDNA sequences. The ITS regions are non-coding sequences interspersed among highly conserved fungal rDNA and have been shown to have high level of heterogeneity among different fungal genera and species. See, e.g., I wen et al., Med. Mycol., 2002, 40:87-109. In some embodiments, the target loci may be one or more ITS (e.g., ITS1, ITS2, ITS3, ITS4, ITS5, and/or ITS6) rDNA sequences. In certain embodiments, the target loci may be one or more sequences from one or more ITS rDNA sequences from the fungal pathogen Fusarium and/or the fungal symbiont Glomus. In some embodiments, the target loci may be one or more ITS rDNA sequences in combination with one or more other gene sequences selected from TEF1, RPB1, RPB2, calmodulin (CaM), beta-tubulin (benA), histone H3 (HIS), nuclear ribosomal intergenic spacer region (IGS rDNA), internal transcribed spacer region (ITS rDNA), nuclear ribosomal RNA large subunit (28S or LSU rDNA) and mitochondrial small subunit (mtSSU rDNA) in fungi.

In some embodiments, the target loci may be one or more conserved viral regions and one or more highly evolving viral regions. For example, in some embodiments, viral target loci may be one or more RdRP (RNA-dependent RNA polymerase) sequences. RdRPs are highly conserved among viruses. See, e.g., Bruenn, Nucleic Acids Res, 2003, 31: 1821-1829. In some embodiments, the target loci may be one or more RdRP sequences in combination with one or more other gene sequences in viruses, such as structural genes (e.g., genes encoding a capsid, envelope, or membrane component), non-structural genes (e.g., genes encoding a polymerase, protease, or integrase), polyprotein genes, non-translated regions, regulators of viral and host gene expression, or genes of unknown function.

In some embodiments, the target loci may be one or more conserved helminth (e.g., nematode) regions and/or one or more highly evolving helminth (e.g., nematode) regions. For example, target loci may be one or more 18S rRNA sequences. 18 rRNA is known to be a common housekeeping sequence in helminths such as nematodes. See, e.g., Floyd et al., Molecular Ecology Notes, 2005, 5:611-612; Hadziavdic et al., PLoS One, 2014, 9(2):e87624. In some embodiments, target loci may be one or more 18S rRNA sequences in combination with one or more other gene sequences in helminths, such as 18S, 28S, ITS, CO1, COX1, or other mitochondrial genes.

In some embodiments, target loci may be sequences from two or more categories or classes as described herein (e.g., from two or more of bacteria, fungi, viruses, nematodes, parasites, and pests). In some embodiments, a plurality of MIPs or a pool of MIPs may be used to target 1, 2, 3, 4, 5, 6, 7, 8, 9, 10, 15, 20, 25, 30, 40, 50, 100 or more species.

In some embodiments, the target locus has one or more mismatches in the sequence complementary to the first and/or the second target locus primers (i.e., the binding sites for the target locus primers) relative to a reference target locus that is entirely complementary to the target locus primer sequence. Thus, in some embodiments, the target locus that is detected and/or quantified has a nucleic acid sequence that is not entirely complementary to the binding site of the first and/or the second target locus primers. In some embodiments, the target locus has 1, 2, 3, 4, 5, or 6 mismatches, relative to a reference target locus sequence, in the first and/or the second target locus primer binding sites. In some embodiments, the target locus has one or two mismatches, relative to a reference target locus sequence, in the first and/or the second target locus primer binding sites.

In some embodiments, target locus primers are designed to anneal to sequences that are highly conserved across species (e.g., “universal” sequences). Without wishing to be bound by theory, it is believed that target locus primers that anneal to sequences that are highly conserved across species (e.g., “universal” sequences) prevent phylogenetic bias.

Exemplary MIP sequences are provided in Table 2.

### Methods of Profiling Microorganisms in an Environmental Sample

The compositions described above are used in methods of profiling microorganisms in an environmental sample, wherein the method includes the steps of:
a) extracting DNA from the environmental sample;
b) denaturing the extracted DNA;
c) incubating the denatured DNA with the specialized MIPs described above under conditions that allow hybridization, thereby generating a sample containing denatured DNA-MIP complexes;
d) after hybridization, performing an extension and ligation reaction that involves incubating the sample containing denatured DNA-MIP complexes with nucleotides, 5′ exo-polymerase lacking strand displacement activity, and a thermostable ligase capable of ligating splinted substrates under conditions that allow extension of the 3′ end of the MIP and ligation to the 5′ end of the MIP;
e) after extension and ligation, incubating the sample containing denatured DNA-MIP complexes with a 3′ to 5′ single strand exonuclease and a 3′ to 5′ double strand exonuclease under conditions sufficient to degrade linear substrates, thereby generating a sample containing circular DNA templates;
f) removing the 3′ to 5′ single strand exonuclease and the 3′ to 5′ double strand exonuclease from the sample containing circular nucleic acid templates;
g) amplifying the circular DNA templates, thereby generating linear DNA containing the sequence of the MIP from 5′ end of the first primer binding site to the 3′ end of the second primer binding site; and
h) sequencing the linear DNA, thereby generating a plurality of sequencing reads containing the sequence of the linear DNA, thereby profiling the microorganisms in the environmental sample.

### Samples

In some embodiments, the sample is an environmental sample (e.g., a soil, dirt, water, air, garbage, or sewage sample). In some embodiments, the sample is a biological sample (e.g., any cells, stool, urine, tissue or bodily fluid obtained from a biological organism, such as blood, serum, plasma, platelets, red blood cells, sputum, saliva, cells, or tissue from kidney, lung, liver, heart, brain, nervous tissue, thyroid, eye, skeletal muscle, cartilage, or bone, etc.). In some embodiments, the sample is a food sample (e.g., vegetable, fruit, fish, dairy, grain, or meat sample). In some embodiments, the sample is a sample that includes plants and/or plant parts.

In some embodiments, the sample includes a mixture of two or more sample types (e.g., two or more environmental samples, biological samples, food samples, and/or samples comprising plants and/or plant parts). In some embodiments, the sample includes a mixture of two or more sample types wherein one or more of the sample types is tagged or barcoded prior to mixing the sample types. In some embodiments, one or more of the sample types is not tagged or barcoded prior to mixing the sample types.

A sample for use in the methods described herein can, in some embodiments, include a mixture of multiple species. In some embodiments, a sample for use in the methods described herein includes material that is known or suspected of containing one or more pathogens (e.g., one or more species of bacteria, phytoplasma, viruses, viroids, rickettsia, fungi, protozoans, helminths, parasites, or pests). In some embodiments, the sample includes one or more bacterial species, phytoplasma species, viral species, viroid species, rickettsia species, fungal species, protozoan species, helminth species, parasite species, and/or pest species that are plant pathogens.

In some embodiments, the sample includes one or more bacterial species. In some embodiments, the bacterial species is a species of Acidovorax, Aeromonas, Agrobacterium, Alicyclobacillus, Anabaena, Anacystis, Acinetobacter, Acidothermus, Arthrobacter, Azobacter, Bacillus, Bifidobacterium, Brevibacterium, Buchnera, Burkholderia, Butyrivibrio, Candidatus, Campestris, Camplyobacter, Clostridium, Clavibacter, Corynebacterium, Chromatium, Coprococcus, Curtobacterium, Dickeya, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Legionella, Ilyobacter, Micrococcus, Microbacterium, Mesorhizobium, Methylobacterium, Methylobacterium, Methylosinus, Methlyosinus trichosporium, Mycobacterium, Neisseria, Pantoea, Pectobacterium, Phytoplasma, Pseudomonas, Prochlorococcus, Ralstonia Rhodobacter, Rhodococcus, Rhodopseudomonas, Rhodopseudomonas, Roseburia, Rhodospirillum, Rhodococcus, Scenedesmus, Sinorhizobium, Sinorhizobium fredii, Streptomyces, Streptococcus, Synecoccus, Saccharomonospora, Staphylococcus, Serratia, Salmonella, Shigella, Spiroplasma, Thermoanaerobacterium, Tropheryma, Tularensis, Temecula, Thermosynechococcus, Thermococcus, Ureaplasma, Xanthomonas, Xylella, Yersinia or Zymomonas.

In some embodiments, the bacterial species is a species of Clavibacter, Xanthomonas, Curtobacterium, Pseudomonas, Acidovorax, Ralstonia, Phytoplasma, Agrobacterium, Xylella, Candidatus, Sinorhizobium, Sinorhizobium fredii, Methylosinus, Methlyosinus trichosporium, or Pectobacterium.

In some embodiments, the sample includes one or more viral species. In some embodiments, the viral species is a species of the viral family Adenoviridae, Arenaviridae, Arteriviridae, Bromoviridae, Bunyaviridae, Caliciviridae, Caulimoviridae, Circoviridae, Closteroviridae, Comoviridae, Coronaviridae, Cystoviridae, Flaviviridae, Geminiviridae, Herpesviridae, Hypoviridae, Iridoviridae, Leviviridae, Myoviridae, Orthomyxoviridae, Paramyxoviridae, Partitiviridae, Parvoviridae, Picornaviridae, Podoviridae, Potyviridae, Poxviridae, Reoviridae, Retroviridae, Rhabdoviridae, Sequiviridae, Siphoviridae, Togaviridae, Tombusviridae, or Totiviridae. In some embodiments, the viral species is a species of DNA virus (e.g., dsDNA virus or ssDNA virus). In some embodiments, the viral species is a species of RNA virus (e.g., dsRNA virus or ssRNA virus). In some embodiments, the viral species is a species of reverse transcribing virus (e.g., retrovirus).

In some embodiments, the sample includes one or more viroid species. In some embodiments, the viroid species is a species of the viroid family Pospiviroidae (e.g., a species of the genus Pospiviroid, Hostuviroid, Cocadviroid, Apscaviroid, or Coleviroid) or Avsunviroidae (e.g., a species of the genus Avsunviroid, Elaviroid, or Pelamoviroid).

In some embodiments, the sample includes one or more rickettsia species. In some embodiments, the rickettsia species is a species of Rickettsia aeschlimannii, R. africae, R. akari, R. asiatica, R. australis, R. canadensis, R. conorii, R. cooleyi, R. felis, R. heilongjiangensis, R. Helvetica, R. honei, R. hulinii, R. japonica, R. massiliae, R. montanensis, R. parkeri, R. peacockii, R. prowazekii, R. rhipicephali, R. rickettsia, R. slovaca, R. tamurae, or R. typhi.

In some embodiments, the sample includes one or more fungal species. In some embodiments, the fungal species is a species of Absidia, Acremonium, Alternaria, Aphanocladium, Arhrinium, Arthrobotrys, Aspergillus, Aurobasidium, Bjerkandera, Botryosphaeria, Botrytis, Cephalosporium, Cercospora, Ceriporiopsis, Chaeotomium, Cladosporium, Cochliobolus, Colletotrichum, Corynascus, Cryphonectria, Cryptococcus, Coprinus, Coriolus, Curvularia, Cylindrocarpon, Didymella, Diplodia, Drechslera, Elsinoe, Endothis, Engyodontium, Epicoccum, Erisiphae, Eurotium, Eutypa, Fairy ring fungi, Fusarium, Gaeumannomyces, Geotrichum, Gibberella, Gliocladium, Glomus, Gonatobotryum, Histoplasma, Humicola, Hypocrea, Leptosphaeria, Macrophomina, Michrodochium, Microsporum, Monilinia, Mucor, Mycosphaerella, Myrothecium, Myxotrichum, Neurospora, Nigrospora, Paecilomyces, Penicillium, Peronospora, Petriella, Peziza, Phaeoacremonuium, Phaeomoniella, Phoma, Phomopsis, Phytophthora, Phytotrichopsis, Pithomyces, Podospora, Phlebia, Piromyces, Pyricularia, Puccinia, Pythium, Rhizoctonia, Rhizomucor, Rhizopus, Schizophyllum, Sclerotinia, Scopulariopsis, Scytalidium, Septoria, Sporothrix, Sporotrichum, Stachybotrys, Stemphylium, Talaromyces, Torula, Trichoderma, Typhula, Ulocladium, Verticillium, Volvariella, or Wallemia.

In some embodiments, the fungal species is a species of Phoma, Alternaria, Mycosphaerella, Colletotrichum, Cercospora, Peronospora, Septoria, Didymella, Verticillium, Fusarium, Glomus, Pyricularia, Cladosporium, Stemphylium, Phytophthora, Botrytis, Cylindrocarpon, 5 Phomopsis, Monilinia, Phaeoacremonuium, Phaeomoniella, Cylindrocarpon, Eutypa, Botryosphaeria, Rhizoctonia, Pythium, Sclerotinia, Michrodochium, Gaeumannomyces, Leptosphaeria, Typhula, Drechslera, Erisiphae, Pyricularia, Puccinia, Fairy ring fungi, Gliocladium, Phytotrichopsis, Elsinoe, or Macrophomina.

In some embodiments, the sample includes one or more helminth species. In some embodiments, the helminth species is a species of Achlysiella, Anguina, Aphelenchoides, Belonolaimus, Bursaphelenchus, Criconemoides, Ditylenchus, Dolichodorus, Globodera, Gracilacus, Helicotylenchus, Hemicriconemoides, Hemicycliophora, Heterodera, Hirschmanniella, Hoplolaimus, Longidorus, Meloidogyne, Merlinius, Mesocriconema, Naccobus, Paralongidorus, Paratrichodorus, Paratylenchus, Pratylenchus, Quinisulcius, Radopholus, Rotylenchulus, Trichodorus, Tylenchorhynchus, Tylenchulus, or Xiphinema.

In some embodiments, the sample includes one or more parasite species. In some embodiments, the parasite is a protozoan (e.g., an amoeba, a flagellate, a ciliate, or a sporozoan), a fungal parasite, a nematode, an ectoparasite (e.g., ticks, fleas, lice, and mites), or a plant parasite.

In some embodiments, the sample includes one or more pest species. In some embodiments, the pest species is a is an insect (e.g., a species of the order Anoplura, Coleoptera, Dermaptera, Diptera, Hemiptera, Hymenoptera, Isoptera, Lepidoptera, Mallophaga, Orthoptera, Psocoptera, Siphonaptera, or Thysanoptera), an arachnid (e.g., a species of the order Acarina), or a nematode (e.g., a species of the genus Anguina, Belonolaimus, Bursaphelenchus, Criconemoides, Ditylenchus, Globodera, Gracilacus, Helicotylenchus, Hemicycliophora, Heterodera, Hirschmanniella, Hoplolaimus, Longidorus, Meloidogyne, Merlinius, Naccobus, Paratrichodorus, Paratylenchus, Pratylenchus, Quinisulcius, Radopholus, Rotylenchulus, Trichodorus, Tylenchulus, or Xiphinema).

In some embodiments, a sample includes a mixture of multiple species (e.g., 2 or more, 10 or more, 100 or more, 1000 or more, 10000 or more, 20000 or more, 30000 or more, 40000 or more, 50000 or more, 60000 or more, 70000 or more, 80000 or more, 90000 or more, 100000 or more, 200000 or more species) from the same class or category, or from two or more classes or categories, as described herein (e.g., species from one of more of the categories of bacteria, viruses, fungi, nematodes, parasites, and/or pests).

Samples can be collected by methods known in the art. For example, samples such as environmental samples can be collected by swabs, wipes, vacuuming, water sampling, or air sampling. In some embodiments, a sample, such as soil, is collected into a container.

In some embodiments, a sample is not subjected to processing prior to being used in the methods of the present invention. In some embodiments, a sample is subjected to one or more processing steps before being used in the methods of the present invention. For example, in some embodiments, a sample is liquefied, fragmented, homogenized, pulverized, crushed, chopped, diluted, concentrated, filtered, pulsified, sonicated, or a combination thereof. Exemplary methods are described in Ausubel et al., Current Protocols in Molecular Biology (1994); Sambrook and Russell, “Fragmentation of DNA by sonication,” Cold Spring Harbor Protocols (2006); and Burden, “Guide to the Homogenization of Biological Samples,” Random Primers (2008), pages 1-14.

Extracting DNA from the Samples

DNA can be extracted from the sample using methods known in the art. In some embodiments, DNA extraction is accomplished by chemical, physical/mechanical, or enzymatic means or a combination thereof.

In some embodiments, the method includes extracting DNA from the sample by chemical means. For example, in some embodiments, cell lysis reagents (e.g., detergents such as sodium dodecyl sulfate or chaotrophic salts such as guanidium thiocyanate) are added to the sample to lyse cells. Optionally a protease (including but not limited to proteinase K) can be used.

In some embodiments, the method includes the use of enzymatic means. For example, in some embodiments, a sample is contacted with an enzyme such as lysozyme that breaks down components of the sample such as cell walls.

In some embodiments, the method includes the use of physical, mechanical, or other means. For example, in some embodiments, a sample is physically sheared (e.g., by sonication), microwave treated, or thermally shocked.

DNA can be isolated from the mixture as is known in the art. In some embodiments, phenol/chloroform extractions are used to separate DNA from proteins and lipids in the sample, and the DNA is subsequently precipitated (e.g., by ethanol, isopropanol, or potassium acetate). In some embodiments, the DNA is subjected to a further purification step. For example, in some embodiments, DNA can be purified using a purification column.

Methods of DNA extraction are described, for example, in Hill et al., Pathogens, 2015, 4:335-354; Robe et al., European Journal of Soil Biology, 2003, 39: 183-190; and in “Environmental Nucleic Acid Extraction,” in ENVIRONMENTAL MICROBIOLOGY, 2005, vol. 397, Leadbetter, J. R., ed, Elsevier Academic Press.

In some embodiments, RNA (e.g., mRNA or viral RNA) is extracted from the environmental sample according to any method known in the art and reverse transcribed into cDNA according to any method known in the art. As used herein, DNA may refer to genomic DNA, extrachromosomal DNA (e.g., plasmids), or cDNA.

### Denaturing the Extracted DNA

In some embodiments, extracted DNA is denatured by incubating at about 95° C. to about 98° C. in the presence of the desired MIP or pool of MIPs. In some embodiments, extracted DNA is denatured by incubating at about 95° C. to about 98° C. for about 3 minutes in the presence of the desired MIP or pool of MIPs. In some embodiments, extracted DNA is denatured by incubating at 95° C., 96° C., 97° C., or 98° C. for about 3 minutes in the presence of the desired MIP or pool of MIPs. In some embodiments, extracted DNA is denatured by incubating at 98° C. for about 3 minutes in the presence of the desired MIP or pool of MIPs.

Incubating the Denatured DNA with MIPs

In some embodiments, denatured DNA extracted from an environmental sample is incubated with one molecular inversion probe (MIP) comprising first and second target locus primers with non-overlapping sequence complementarity to a first target locus under conditions that allow hybridization. In some embodiments, denatured DNA extracted from an environmental sample is further incubated with a second MIP comprising first and second target locus primers with non-overlapping sequences complementary to a second target locus.

In some embodiments, denatured DNA extracted from an environmental sample is further incubated with a plurality or a pool of MIPs (e.g., 2, 3, 4, 5, 10, 25, 50, 100, 1000, 2000, or more MIPs), wherein each MIP includes first and second target locus primers with non-overlapping sequences complementary to a target locus.

In some embodiments, a single molecular work flow is used to profile microorganisms in an environmental sample according to the methods disclosed herein. A “single molecular work flow” refers to the multiplexing of many different MIPs in a single reaction, as opposed to running dozens or hundreds of individual reactions with different MIPs.

In some embodiments, each MIP in a plurality or pool of MIPs includes first and second target locus primers with non-overlapping sequences complementary to the same target locus. In some embodiments, each MIP in a plurality or pool of MIPs includes first and second target locus primers with non-overlapping sequences complementary to: the same nucleic acid sequences on the same target locus, partially overlapping nucleic acid sequences on the same target locus, or non-overlapping nucleic acid sequences on the same target locus.

In some embodiments, each MIP in a plurality or pool of MIPs includes first and second target locus primers with non-overlapping sequence complementarity to different target loci. In some embodiments, a plurality of MIPs or a pool of MIPs may be used to target 1 or more, 2 or more, 3 or more, 4 or more, 5 or more, 6 or more, 7 or more, 8 or more, 9 or more, 10 or more, 15 or more, 20 or more, 25 or more, 30 or more, 40 or more, 50 or more, 100 or more, 1000 or more, 2000 or more, 5000 or more, 10000 or more, or 100000 or more target loci.

Non-limiting examples of target loci are provided in the “Target Loci” section above.

Non-limiting examples of organisms include those provided in the “Samples” section above.

### Hybridization

In some embodiments, the conditions that allow hybridization of denatured DNA and a MIP or pool of MIPs include gradually reducing (i.e., ramp-down) the temperature of the denatured DNA and MIP or the pool of MIPs from the temperature used for denaturation (about 95° C. to about 98° C.) to a temperature of between about 50° C. to about 55° C. In some embodiments, the conditions that allow hybridization of denatured DNA and a MIP or pool of MIPs include gradually reducing (i.e., ramp-down) the temperature of the denatured DNA and MIP or the pool of MIPs from the temperature used for denaturation (about 95° C. to about 98° C.) to a temperature of about 55° C. In some embodiments, the conditions that allow hybridization of denatured DNA and a MIP or pool of MIPs further include incubating the denatured DNA and the MIP or pool of MIPs at a temperature of about 50° C., about 51° C., about 52° C., about 53° C., about 54° C., or about 55° C. for about 4 hours. In some embodiments, the conditions that allow hybridization of denatured DNA and a MIP or pool of MIPs further include incubating the denatured DNA and the MIP or pool of MIPs at a temperature of about 55° C. for about 4 hours. Following incubation of the denatured DNA and the MIP or pool of MIPs under conditions that allow hybridization of denatured DNA and a MIP or pool of MIPs, a sample comprising denatured DNA-MIP complexes is generated.

### Extension and Ligation

As used herein, the term “extension and ligation reaction” refers to a reaction in which a gap is filled by the action of a polymerase and a ligase between 5′ and 3′ ends of a MIP that is hybridized to a complementary target nucleic acid (e.g., target locus). In some embodiments, the filled gap is more than one nucleotide, e.g., 2, 3, 4, 5, 6, 7, 8, 9, 10, 15, 20, 25, 30, 40, 50 75, 100, 150, 200, 250 or more nucleotides, e.g., between a first sequence in a target locus complimentary to a first target locus primer and a second sequence in a target locus complimentary to a second target locus primer.

In some embodiments, the polymerase in the extension and ligation reaction is a 5′exo-polymerase lacking strand displacement activity. For example, without limitation, the 5′exo-polymerase lacking strand displacement activity may be Stoffel fragment, TaqIT, Klenow Large Fragment, or Phusion polymerase.

In some embodiments, the ligase in the extension and ligation reaction is a thermostable ligase that acts on splinted substrates. For example, without limitation, the thermostable ligase that acts on splinted substrates may be Taq ligase, T4 DNA ligase, or Ampligase.

The extension and ligation reaction is carried out under conditions that allow extension of the 3′ end of the MIP by the polymerase and ligation to the 5′ end of the MIP by the ligase. In certain embodiments, conditions that allow extension of the 3′ end of the MIP by the polymerase and ligation to the 5′ end of the MIP by the ligase include a reaction mixture comprising dNTPS, appropriate buffers (e.g., 2× fusion master mix), a 5′exo-polymerase lacking strand displacement activity, and a thermostable ligase that acts on splinted substrates. In certain embodiments, conditions that allow extension of the 3′ end of the MIP by the polymerase and ligation to the 5′ end of the MIP by the ligase include incubating the extension and ligation reaction at a temperature of between about 50° C. to about 55° C. for about 30 minutes to about 90 minutes.

### Removal of Linear DNA

In some embodiments, following the extension and ligation reaction, linear DNA is removed from the sample containing denatured DNA-MIP complexes, thereby enriching for target loci. “Enriching” refers to increasing the relative abundance of a target molecule (e.g., target locus) in a population of molecules. For example, increasing the relative abundance of target loci in a population of nucleic acids.

In some embodiments, the sample comprising denatured DNA-MIP complexes is incubated with a 3′ to 5′ single strand exonuclease and a 3′ to 5′ double strand exonuclease under conditions sufficient to degrade linear substrates, thereby generating a sample comprising circular DNA templates.

In some embodiments, the sample containing denatured DNA-MIP complexes is incubated with a first exonuclease under conditions sufficient to degrade linear substrates and subsequently with a second exonuclease under conditions sufficient to degrade linear substrates. In some embodiments, the first exonuclease is removed from the sample prior to incubating the sample containing denatured DNA-MIP complexes with the second exonuclease. In some embodiments, the first exonuclease is a 3′ to 5′ single strand exonuclease and the second exonuclease is a 3′ to 5′ double strand exonuclease. In some embodiments, the first exonuclease is a 3′ to 5′ double strand exonuclease and the second exonuclease is a 3′ to 5′ single strand exonuclease.

As will be readily apparent to those of skill in the art, a 3′ to 5′ single strand exonuclease is any exonuclease with 3′ to 5′ single strand exonuclease activity. A non-limiting example of a 3′ to 5′ single strand exonucleases is exonuclease I.

As will be readily apparent to those of skill in the art, a 3′ to 5′ double strand exonuclease is any exonuclease with 3′ to 5′ double strand exonuclease activity. Non-limiting examples of a 3′ to 5′ double strand exonucleases include exonuclease III and Kamchatka crab nuclease.

### Removal of Exonucleases

In some embodiments, exonucleases are removed by heating the sample (i.e., heat inactivation). In some embodiments, exonucleases are removed by heating the sample (i.e., heat inactivation) to a temperature of about 80° C. for about 20 minutes. In some embodiments, exonucleases are removed by purification of the DNA. Purification of DNA may be performed using any method known in the art.

### Amplification and Sequencing Library Preparation

“Amplification” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing.

In some embodiments, the amplification step and preparation of the sequencing library are performed in the same reaction.

In some embodiments, the step of amplifying includes a PCR reaction. In some embodiments, the PCR reaction includes a PCR reaction mix, wherein the PCR reaction mix includes a high-fidelity proofreading polymerase and sequencing primers (e.g., as described in further detail below).

High-fidelity proofreading polymerases that can be used in the methods of the present disclosure include, without limitation, Phusion, Taq polymerase, AccuPrime polymerase, Kapa HiFi, NEB Q5, or Pfu.

Amplification of nucleic acids can be performed under temperature cycled or isothermal conditions, or combined conditions. Amplification can be linear or exponential. Many well-known methods of nucleic acid target amplification require thermocycling to alternately denature double-stranded DNA and hybridize primers; however, other well-known methods of nucleic acid amplification are isothermal.

In some embodiments, the amplification step includes polymerase chain reaction (PCR), quantitative PCR, or real-time PCR. Quantitative amplification (including, but not limited to, real-time PCR) methods allow for determination of the amount of the species targets (and optionally spike-ins) that are present in a sample. The polymerase chain reaction (PCR) (Mullis, 1987 U.S. Pat. No. 4,683,202; Saiki et al., 1985, Science (New York, N.Y.), 230(4732), 1350-1354), uses multiple cycles of denaturation, annealing of primer pairs to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA (Gelfand et al., “Reverse Transcription with Thermostable DNA Polymerases—High Temperature Reverse Transcription,” (Gelfand, 1994, U.S. Pat. No. 5,322,770; Gelfand & Myers, 1994, U.S. Pat. No. 5,310,652).

Quantitative amplification methods (e.g., quantitative PCR or quantitative linear amplification) involve amplification of nucleic acid template, directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mal Biotechnol. 20(2): 163-79 (2002). Amplifications can be monitored in “real time.”

Other amplification methods include but are not limited to the ligase chain reaction method (LCR) (Laffler et al., 1993 Annales De Biologie Clinique, 51(9), 821-826), strand displacement amplification (SDA) (Walker et al., 1993, U.S. Pat. No. 5,270,184; Walker, 1995, U.S. Pat. No. 5,455,166; Walker et al., 1992, Nuc Acids Res, 20(7), 1691-1696; Walker, 1992, PNAS, 89(1), 392-396), thermophilic SDA (tSDA) (Fraiser et al., 2002, European Pat. No. 0684315), nucleic acid sequence based amplification (NASBA) (Compton, 1991, Nature, 350(6313), 91-92; Malek, et al., 1992, Clin Chem 38:458), RNA replicase-mediated amplification (Lizardi et al., 1988, Nat Biotech, 6(10), 1197-1202), transcription-based amplification (Kwoh et al., 1989, PNAS, 86(4), 1173-1177), self-sustained sequence replication (3SR), (Guatelli et al., 1990, PNAS, 87(5), 1874-1878; Landgren (1993) Trend Gen 9:199-202; Lee et al., Nucleic Acid Amplification Technologies (1997)), transcription-mediated amplification (TMA) (Kwoh et al., 1989, PNAS, 86(A), 1173-1177; Kacian & Fultz, 1995, U.S. Pat. No. 5,480,784; Kacian & Fultz, 1996, U.S. Pat. No. 5,399,491), rolling circle amplification (RCA) (Fire & Xu, 1995, PNAS, 92(10), 4641-4645; Lizardi, 1998, U.S. Pat. No. 5,854,033), nucleic acid amplification using nicking agents (Van Ness et al., 2006, U.S. Pat. No. 7,112,423), nicking and extension amplification reaction (NEAR) (Maples et al., 2009, U.S. Pat. Application No. 2009/0017453A1), helicase dependent amplification (HDA) (Kong et al., 2004, U.S. Pat. Application No. 2004/0058378A1; Kong et al., 2007 U.S. Pat. Application No. 2007/0254304 A1), quadruplex priming amplification (Adams et al., Analyst, 2014, 139, 1644-1652), Expar amplification (Van Ness et al., PNAS, 2003 100(8):4504-4509), cross priming amplification (Xu et al., Sci Rep. 2012; 2: 246), SMAP amplification (Mitani et al., Nat Methods. 2007; 4(3):257-62), multiple displacement amplification (MDA) (Hutchison et al., PNAS, 2005, 102 (48): 17332-6), recombinase polymerase amplification (RPA) (Euler et al., J Clin Virol, 2012, 54(4): 308-12), and single primer isothermal amplification (SPIA) (Kum et al., Clin Chem, 2005 51(10):1973-1981). For further discussion of known amplification methods see Persing, 1993, “In Vitro Nucleic Acid Amplification Techniques” in Diagnostic Medical Microbiology: Principles and Applications (Persing et al., Eds.), pp. 51-87 (American Society for Microbiology, Washington, D.C.).

### Sequencing Primers

In certain embodiments, sequencing primers refer to primers for use during amplification and library preparation. In certain embodiments, sequencing primers refer to primers for use in an amplification reaction. In certain embodiments, sequencing primers refer to primers for use in a PCR reaction (i.e., PCR primers). In some embodiments, the sequencing primers include a sequence complementary to the first and second sequencing primer binding sites on the universal backbone. In some embodiments, the sequencing primers further include an adaptor or fragment thereof for a sequencing platform (e.g., a P5 or P7 adaptor, a partial P5 or P7 adaptor for Illumina sequencing). In certain embodiments, the sequencing primers include, from the 5′ end to the 3′ end, an adaptor or fragment thereof for a sequencing platform and sequence complementary to a sequencing primer binding site on the universal backbone.

In some embodiments, the sequencing primers further include sample index. A sample index identifies a sequencing read as corresponding to a specific sample and can be used to identify a sample or source of the DNA. Thus, when DNA is derived from multiple sources (e.g., different soil samples), the DNA from sample can be tagged with different sample indexes such that the source of the sample can be identified. Sample indexes are also referred to as sample tags or sample barcodes. Any suitable sample index or set of sample indexes can be used in the methods of the present disclosure, as known in the art and as exemplified by the disclosures of U.S. Pat. No. 8,053,192 and PCT Publication No. WO05/068656, which are incorporated herein by reference in their entireties.).

In certain embodiments, the sequencing primers include, from the 5′ end to the 3′ end, an adaptor or fragment thereof for a sequencing platform, a sample index, and sequence complementarity to a sequencing primer binding site on the universal backbone.

Exemplary sequencing primer sequences are provided in Table 3. In addition, Exemplary sequencing primer sequences included sample index sequences are provided in Table 4.

Sequencing

Any technique for sequencing DNA known to those skilled in the art can be used in the methods of the present disclosure. Non-limiting examples of nucleotide sequencing include Sanger sequencing, capillary array sequencing, thermal cycle sequencing (Sears et al., Biotechniques 13:626-633 (1992)), solid-phase sequencing (Zimmerman et al., Methods Mal. Cell Biol. 3:39-42 (1992)), sequencing with mass spectrometry such as matrix-assisted laser desorption/ionization time-of-flight mass spectrometry (MALDI-TOF/MS; Fu et 5 al., Nature Biotech. 16:381-384 (1998)), and sequencing by hybridization (Chee et al., Science 274:610-614 (1996); Drmanac et al., Science 260: 1649-1652 (1993); Drmanac et al., Nature Biotech. 16:54-58 (1998)).

In some embodiments, “next generation sequencing” methods can be used, for example but not limited to, sequencing by synthesis (e.g., HiSeq™, MiSeq™, or Genome Analyzer, each available from Illumina, Inc.), sequencing by ligation (e.g., SOLiD™, Life 10 Technologies), ion semiconductor sequencing (e.g., Ion Torrent™, Life Technologies), and pyrosequencing (e.g., 454™ sequencing, Roche Diagnostics). In some embodiments, nucleotide sequencing includes high-throughput sequencing. In high-throughput sequencing, parallel sequencing reactions using multiple templates and multiple primers allows rapid sequencing of genomes or large portions of genomes. See, e.g., WO 03/004690, WO 03/054142, WO15 2004/069849, WO 2004/070005, WO 2004/070007, WO 2005/003375, WO 2000/006770, WO2000/027521, WO 2000/058507, WO 2001/023610, WO 2001/057248, WO 2001/057249, WO2002/061127, WO 2003/016565, WO 2003/048387, WO 2004/018497, WO 2004/018493, WO2004/050915, WO 2004/076692, WO 2005/021786, WO 2005/047301, WO 2005/065814, WO2005/068656, WO 2005/068089, WO 2005/078130, Seo, et al., Proc. Natl. Acad Sci. USA20 (2004) 101:5488-5493; and Liu et al., J Biomed Biotechnol, 2012, 2012:251364.

In some embodiments, nucleotide sequencing includes sequencing by synthesis. In some embodiments, nucleotide sequencing includes massively parallel sequencing using reversible chain termination, for example as available from Illumina, Inc. In sequencing by synthesis, a fluorescently labeled reversible terminator is imaged as each dNTP is added, and then cleaved to allow incorporation of the next base. The sequencing process produces a set of nucleic acid sequence reads of uniform length. The sequencing reaction can be conducted simultaneously on thousands or millions or different template molecules on a solid surface. Methods of sequencing by synthesis are known in the art. See, e.g., Bronner et al., Curr Protoc Hum Genet, 2009 July; doi: 0.1002/0471142905.hg10802s62; Rohland et al., Genome Research, 2012, 22:939-946. One method of sequencing by synthesis utilizes “bridge amplification” to enable the detection of the fluorescent labels. Briefly, a nucleic acid sample is prepared by fragmenting the nucleic acid into fragments of about 200 bases in length and adding adapters to each end. The library of fragments is flowed across a solid surface (flowcell) and the template fragments bind to the surface. A solid phase bridge amplification PCR process, in which individual templates in the library bend and bridge to another complementary oligonucleotide on the flowcell surface in repeated denaturation and extension cycles, creates approximately one million copies of each template in physical clusters on the flowcell surface.

In some embodiments, nucleotide sequencing includes single-molecule, real-time (SMRT) sequencing (for example, as available from Pacific Biosciences). SMRT sequencing is a process by which single DNA polymerase molecules are observed in real time while they catalyze the incorporation of fluorescently labeled nucleotides complementary to a template nucleic acid strand. Methods of SMRT sequencing are known in the art and were initially described by Flusberg et al., Nature Methods, 7:461-465 10 (2010), which is incorporated herein by reference for all purposes. Briefly, in SMRT sequencing, incorporation of a nucleotide is detected as a pulse of fluorescence whose color identifies that nucleotide. The pulse ends when the fluorophore, which is linked to the nucleotide's terminal phosphate, is cleaved by the polymerase before the polymerase translocates to the next base in the DNA template. Fluorescence pulses are characterized by emission spectra as well as by the duration of the pulse (“pulse width”) and the interval between successive pulses (“interpulse duration” or “IPD”). Pulse width is a function of all kinetic steps after nucleotide binding and up to fluorophore release, and IPD is a function of the kinetics of nucleotide binding and polymerase translocation. Thus, DNA polymerase kinetics can be monitored by measuring the fluorescence pulses in SMRT sequencing.

In addition to measuring differences in fluorescence pulse characteristics for each fluorescently-labeled nucleotide (i.e., adenine, guanine, thymine, and cytosine), differences can also be measured for non-methylated versus methylated bases. For example, the presence of a methylated base alters the IPD of the methylated base as compared to its non-methylated counterpart (e.g., methylated adenosine as compared to non-methylated adenosine). Additionally, the presence of a methylated base alters the pulse width of the methylated base as compared to its non-methylated counterpart (e.g., methylated cytosine as compared to nonmethylated cytosine) and furthermore, different modifications have different pulse widths (e.g., 5-hydroxymethylcytosine has a more pronounced excursion than 5-methylcytosine). Thus, each type of non-modified base and modified base has a unique signature based on its combination of IPD and pulse width in a given context. The sensitivity of SMRT sequencing can be further enhanced by optimizing solution conditions, polymerase mutations and algorithmic approaches that take advantage of the nucleotides' kinetic signatures, and deconvolution techniques to help resolve neighboring methylcytosine bases.

In some embodiments, nucleotide sequencing includes nanopore sequencing (for example, as available from Oxford Nanopore Technologies (ONT)). Nanopore sequencing is a process by which a polynucleotide or nucleic acid fragment is passed through a pore (such as a protein pore) under an applied potential while recording modulations of the ionic current passing through the pore. Methods of nanopore sequencing are known in the art; see, e.g., Clarke et al., Nature Nanotechnology 4:265-270 (2009), which is incorporated herein by reference for all purposes. Briefly, in nanopore sequencing, as a single-stranded DNA molecule passes through a protein pore, each base is registered, in sequence, by a characteristic decrease in current amplitude which results from the extent to which each base blocks the pore. An individual nucleobase can be identified on a static strand, and by sufficiently slowing the rate of speed of the DNA translocation (e.g., through the use of enzymes) or improving the rate of DNA capture by the pore (e.g., by mutating key residues within the protein pore), an individual nucleobase can also be identified while moving.

In some embodiments, nanopore sequencing includes the use of an exonuclease to liberate individual nucleotides from a strand of DNA, wherein the bases are identified in order of release, and the use of an adaptor molecule that is covalently attached to the pore in order to permit continuous base detection as the DNA molecule moves through the pore. As the nucleotide passes through the pore, it is characterized by a signature residual current and a signature dwell time within the adapter, making it possible to discriminate between nonmethylated nucleotides. Additionally, different dwell times are seen between methylated nucleotides and the corresponding non-methylated nucleotides (e.g., 5-methyl-dCMP has a longer dwell time than dCMP), thus making it possible to simultaneously determine nucleotide 25 sequence and whether sequenced nucleotides are modified. The sensitivity of nanopore sequencing can be further enhanced by optimizing salt concentrations, adjusting the applied potential, pH, and temperature, or mutating the exonuclease to vary its rate of processivity.

In some embodiments, the method of detecting the sequence and amount of the species targets and spike-ins includes deep sequencing. Deep sequencing refers to sequencing nucleic acid sequences (e.g., a region of a genome) multiple times, even as many as hundreds or thousands of times. Typically, deep sequencing uses high-throughput sequencing methods to generate a large number of reads (e.g., hundreds to thousands of reads) at a given position. After sequencing, reads are aligned, such as by multiple sequence alignment or by alignment to a reference. Following alignment, the sequence reads are analyzed (e.g., to identify variants from aligned reads). Deep sequencing allows for the detection of rare components in a sample (e.g., cells that occur in a sample at low frequency or rare nucleic acid variants in a population of nucleic acids). In some embodiments, targeted deep sequencing of specific nucleotide sequences (e.g., particular genes of interest) is used to identity rare variants (e.g., variants that occur in less than 1% of a sample population). Methods of deep sequencing are known in the art. See, e.g., Mardis, Ann. Rev. Genomics Hum. Genet., 2008, 9:387-402; McElroy et al., Microbial Informatics and Experimentation, 2014, 4: 1 (doi: 10. 1 186/2042-5783-4-1); Schmitt et al., PNAS, 2012, 109:14508-14513.

In some embodiments, the sequencing step includes deep sequencing. In some embodiments, the sequencing step includes deep sequencing a target locus region (e.g., target gene or target genomic region) and determining the relative abundance in the population of a mutation or variant at the target locus region (e.g., a mutation in or rare variant of a bacterial species, phytoplasma species, viral species, viroid species, rickettsia species, fungal species, helminth species, protozoan, parasite species, and/or pest species).

In some embodiments, a single environmental sample is sequenced in one sequencing run. In some embodiments, two or more environmental samples are sequenced simultaneously in one sequencing run. In some embodiments, the step of amplifying includes the use of sequencing primers comprising a sample index.

In some embodiments, the sequencing step includes pore-based sequencing. In some embodiments, the sequencing step includes loop sequencing.

In some embodiments, the sequencing step includes single-end sequencing. In some embodiments, the sequencing step includes paired-end sequencing.

In certain embodiments, the sequencing technique used in the methods disclosed herein generates a plurality of reads. For example, the sequencing technique used in the methods disclosed herein may generate a plurality of reads (e.g., at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, at least 1,000,000 reads per run, at least 2,000,000 reads per run, at least 3,000,000 reads per run, at least 4,000,000 reads per run at least 5000,000 reads per runs at least 6,000,000 reads per run at least 7,000,000 reads per run at least 8,000,000 reads per runs at least 9,000,000 reads per run, or at least 10,000,000 reads per run).

### Data Analysis

In some embodiments, the method includes identifying and/or quantifying a wild-type target locus sequence that is entirely complementary to the target locus primer binding sequence (e.g., nucleotide sequencing and/or quantifying the amount of the wild-type target sequence). In some embodiments, the method includes detecting and/or quantifying (i) a wild-type target sequence that is entirely complementary to the target locus primer binding sequence, and (ii) a mutated target locus sequence that is not entirely complementary to the target locus primer binding sequence. In some embodiments, the method further includes comparing the amount of the mutated target locus sequence in the sample to the amount of the wild-type target locus sequence in the sample.

### Grouping Reads by Index

In some embodiments, one, two or more environmental samples are sequenced simultaneously in one sequencing run. In some embodiments, the step of amplifying includes the use of sequencing primers comprising a sample index (e.g., as described above). A sample index identifies a sequencing read as corresponding to a specific sample.

In some embodiments, sequencing reads are grouped if they include the same sample index sequence, thereby generating bins comprising sequencing reads from the same sample.

### Grouping Reads by UMI Sequence to Quantify Unique Target Loci in the Sample

As used herein, the term “unique target locus” refers to a unique a target locus that was present in the environmental sample. For example, if an environmental sample contains a single copy of a target locus “A”, then there is one unique target locus “A” in the environmental sample. In another example, if an environmental sample contains 10 cells, each carrying one copy of target locus “B”, then there are 10 unique target loci “B”. The term may be used interchangeably with the terms “unique target molecule”, “unique progenitor molecule”, “progenitor molecule”, “unique progenitor target locus”, “progenitor target locus”, and the like, unless otherwise specified.

In some embodiments, sequencing reads from the same sample are grouped if they include the same UMI sequence, thereby generating bins comprising sequencing reads from the same sample and with the same UMI sequence. In some embodiments, the number of bins comprising sequencing reads from the same sample and with the same UMI sequence is enumerated, thereby determining the number of unique target loci in the sample.

### Alignment and Identification of Microorganisms in the Sample

In some embodiments, alignment of sequencing reads to reference sequences can be done manually. In some embodiments, alignment of sequencing reads to reference sequences can be done by a computer algorithm. One example of a computer algorithm for sequence alignment includes, without limitation, the Efficient Local Alignment of Nucleotide Data (ELAND) from the IlluminaGenomics Analysis pipeline. Alternatively, a Bloom filter or similar set membership tester may be employed to align reads to reference genomes. See U.S. Patent Application No. 61/552,374 filed Oct. 27, 2011 which is incorporated herein by reference in its entirety. In some embodiments, the sequencing reads mapped to a reference sequence have 100% sequence complementarity to the reference sequence. In certain embodiments, the sequencing reads mapped to a reference sequence have less than 100% sequence complementarity to the reference sequence.

In some embodiments, alignment of sequencing reads to reference sequences can be done using any method known in the art. Non-limiting examples of methods for alignment of sequencing reads to reference sequences include QIIME, Kraken2, and BWA (exact aligner).

In some embodiments, alignment of the sequencing reads is performed prior to the steps of grouping the sequencing reads by sample index sequence and UMI sequence, and the step of analyzing the sequencing reads from the sample and target locus to determine whether the reads have the same or different nucleotide at each position. In some embodiments, alignment of the sequencing reads is performed after the steps of grouping the sequencing reads by sample index sequence and UMI sequence, and the step of analyzing the sequencing reads from the sample and target locus to determine whether the reads have the same or different nucleotide at each position.

In some embodiments, sequencing reads are aligned to a collection of functional and phylogenetic reference sequences (e.g., as described below in the “Reference Sequences” section), thereby identifying the microorganisms in the environmental sample. In some embodiments, a sequencing read is identified as corresponding to (i.e., mapping or aligning to) a reference sequence if the sequencing read sequence has sequence complementarity to a sequence in the reference sequence. In some embodiments, the sequence complementarity is 100%. In some embodiments, the sequence complementarity is less than 100%. In some embodiments, the reference sequence is as described in the “Reference Sequences” section below. In some embodiments, a microorganism is identified in an environmental sample if at least one sequencing read aligns to a reference sequence from the microorganism. In some embodiments, a microorganism is identified in an environmental sample if one or more sequencing reads align to a reference sequence from the microorganism.

### Reference Sequences

In some embodiments, reference sequences may be functional reference sequences. In some embodiments, reference sequences may be phylogenetic reference sequences. In some embodiments, reference sequences may be genomes of bacterial, fungal, and viral species, as well as chromosomes, extra-chromosomal elements (e.g., plasmids), sub-chromosomal regions (such as strands) thereof. In some embodiments, reference sequences may be genomes, chromosomes, extra-chromosomal elements (e.g., plasmids), or sub-chromosomal regions (such as strands) of one or more of the species listed in the “Samples” section above. In some examples, references include genomes, chromosomes, extra-chromosomal elements, and sub-chromosomal regions of any species.

### Error-Correction

In some embodiments, sequencing reads from the same sample and with the same UMI are analyzed to determine whether the sequencing reads have the same or different nucleotide at each position. In some embodiments, if sequencing reads from the same sample with the same UMI have a different nucleotide relative to each other at one or more positions, it may be determined that the nucleotide difference(s) is an error. In some embodiments, 3 or more sequencing reads from the same sample and with same UMI are analyzed to determine whether the sequencing reads have the same or different nucleotide at each position. In some embodiments, if a first sequencing read has a different nucleotide at one or more positions relative to 2 other sequencing reads from the same sample and with the same UMI, it is determined that the nucleotide difference(s) is an error in the first sequencing read. In some embodiments, the error may be an amplification error. In some embodiments, the error may be a PCR error. In some embodiments, the error may be a sequencing error (e.g., a base calling error).

### Variant Analysis

As used herein, the term “sequence variant” refers to a difference in nucleotide sequence in a target locus relative to a reference sequence, wherein the difference existed in the environmental sample and is not due to a PCR and/or sequencing error.

In some embodiments, sequencing reads from the same sample and target locus are aligned to a reference sequence (e.g., as described in the “Alignment and Identification of Microorganisms in the Sample” section above) and analyzed to determine whether each sequencing read has the same or different nucleotide at each position relative to the reference sequence. In some embodiments, if all sequencing reads from the same sample and target locus have the same nucleotide difference at one or more positions relative to the reference sequence, it may be determined that the nucleotide difference is a sequence variant. In some embodiments, if sequencing reads from the sample and with the same UMI have a different nucleotide at one or more positions relative to the reference sequence, it may be determined that the nucleotide difference is a sequence variant. In some embodiments, if 3 or more sequencing reads from the sample and with the same UMI have a different nucleotide at one or more positions relative to the reference sequence, it is determined that the nucleotide difference is a sequence variant.

### Quantification of Microbial Abundance in the Sample

In some embodiments, microbial abundance in the environmental sample is determined based on the number of unique target loci in the environmental sample. In some embodiments, the microbial abundance in the environmental sample is determined based on the number of unique target loci in the environmental sample corresponding to one or more bacterial species, phytoplasma species, viral species, viroid species, rickettsia species, fungal species, protozoan species, helminth species, parasite species, and/or pest species that are plant pathogens. In some embodiments, the abundance of one or more of the species provided in the “Samples” section above is determined.

In some embodiments, the abundance of one or more bacterial species (e.g., one or more of the species provided in the “Samples” section above) in the environmental sample is determined based on the number of unique 16S rRNA loci in the environmental sample corresponding to each bacterial species.

In some embodiments, the abundance of one or more fungal species (e.g., one or more of the species provided in the “Samples” section above) in the environmental sample is determined based on the number of unique 18S rRNA loci in the environmental sample corresponding to each fungal species. In some embodiments, the abundance of one or more fungal species (e.g., one or more of the species provided in the “Samples” section above) in the environmental sample is determined based on the number of unique ITS region loci in the environmental sample corresponding to each fungal species. In some embodiments, the ITS region locus is the ITS2 gene.

Determination of Chemical Availability and/or Transformation Process Rates

“Transformation Process Rates” refer to rates of chemical transformations in an environmental sample. For example, in certain embodiments, the Transformation Process Rate of denitrification, which refers to the process of converting nitrate to nitrous oxide and dinitrogen gas, is determined. Denitrification is of interest in agriculture due to its function in driving loss of added nitrogen fertilizer. In certain embodiments, Transformation Process Rates are determined based on the number of unique target loci corresponding to enzymes that catalyze chemical transformation reactions. In certain embodiments, determination of Transformation Process Rates reflects potential rates of chemical transformations in an environmental sample.

Chemical availability refers to the amount of usable chemicals in an environmental sample. For example, in certain embodiments, chemical availability refers to the amount of easily usable carbon (e.g., in the form of sugars) that is available for microbial decomposition. In certain embodiments, chemical availability is determined based on the number of unique target loci corresponding to genes that use a given chemical as a substrate. For example, in certain embodiments, the chemical availability of carbon is determined based on the number of unique target loci corresponding to genes that use carbon (e.g., in the form of sugars) as a substrate. In certain embodiments, determination of chemical availability reflects the relative availability of chemicals in an environmental sample. For example, in certain embodiments, the chemical availability of carbon reflects the relative availability of sugars in an environmental sample.

In some embodiments, chemical availability and/or Transformation Process Rates in the environmental sample are determined based on the number of unique target loci and the microorganisms identified in the environmental sample. In some embodiments, chemical availability and/or Transformation Process Rates in the environmental sample are determined based on the number of unique target loci. In some embodiments, the unique target loci are one or more of the target loci provided in the “Target Loci” section above. In some embodiments, chemical availability and/or Transformation Process Rates in the environmental sample are determined based on the microorganisms identified in the sample. In some embodiments, the microorganisms in the sample are identified as described in the “Alignment and Identification of Microorganisms in the Sample” section above. In some embodiments, the microorganisms identified in the environmental sample are one or more bacterial species, phytoplasma species, viral species, viroid species, rickettsia species, fungal species, protozoan species, helminth species, parasite species, nematode species, and/or pest species that are plant pathogens. In some embodiments, the microorganisms identified in the environmental sample are one or more of the species provided in the “Samples” section above.

### Spike-Ins

In some embodiments, a known amount of a spike-in is added to the environmental sample to determine the absolute microbial abundance and/or the absolute number of unique target loci in the environmental sample.

In some embodiments, the spike-in is a known amount of cells or organisms. In some embodiments, the spike-in is a known amount of one or more of bacterial cells, fungal cells, or viral particles. In some embodiments, the spike-in is a known amount of one or more of the species provided in the “Samples” section above (e.g., 1, 2, 3, 4, 5, 6, 7, 8, 9, 10, 15, 20, 25, 30, 40, 50 or more different species). In some embodiments, the spike-in is added to the environmental sample prior to the step of extracting DNA from the environmental sample. In some embodiments, the “known amount” of the spike-in is determined by reference to cell or viral particle count. In some embodiments, the amount of a spike-in that is added to the sample is about 1, about 5, about 10, about 50, about 100, about 200, about 300, about 400, about 500, about 600, about 700, about 800, about 900, about 1000, about 1500, about 2000, about 3000, about 4000, about 5000, about 10,000 (10⁴), about 10⁵, about 10⁶, about 10⁷, about 10⁸, about 10⁹, about 10¹⁰, or more cells or viral particles. In some embodiments, the amount of a spike-in is measured in cells or viral particles per unit volume (e.g., cells or viral particles per ml, cells or viral particles per μl, or cells or viral particles per nl). In some embodiments, the amount of a spike-in that is added to the sample is from about 1 cell or viral particle/ml to about 10¹⁰ cells or viral particles/ml (e.g., about 10² cells or viral particles/ml to about 10¹⁰ cells or viral particles/ml, about 10³ cells or viral particles/ml to about 10⁹ cells or viral particles/ml, or about 10⁴ cells or viral particles/ml to about 10⁸ cells or viral particles/ml). Cell or viral particle count and cells or viral particles per unit volume can be determined by any of a number of methods known in the art.

In some embodiments, the spike-in is a known amount of one or more of a DNA construct, synthetic DNA, or a DNA fragment (e.g., 1, 2, 3, 4, 5, 6, 7, 8, 9, 10, 15, 20, 25, 30, 40, 50 or more DNA constructs, synthetic DNA, or a DNA fragments). In some embodiments, the spike-in corresponds to a target locus. In some embodiments, the spike-in is a target locus from one or more of the species provided in the “Sample” section above (e.g., 1, 2, 3, 4, 5, 6, 7, 8, 9, 10, 15, 20, 25, 30, 40, 50 or more different species). In some embodiments, the spike-in is added to the environmental sample after the step of extracting DNA from the environmental sample. In some embodiments, the “known amount” of the spike-in is determined by reference to molar quantity. In some embodiments, amount of a spike-in that is added to the sample is about 0.001 pM to about 1 mM, about 0.01 pM to about 1 mM, about 0.1 pM to about 1 mM, about 1 pM to about 1 mM, about 1 nM to about 1 mM, about 100 nM to about 1 mM, 1 nM to about 100 μM, about 1 nM to about 1000 nM, or about 500 nM to about 50 μM (e.g., about 1 nM, about 10 nM, about 50 nM, about 100 nM, about 200 nM, about 300 nM, about 400 nM, about 500 nM, about 600 nM, about 700 nM, about 800 nM, about 900 nM, about 1 μM, about 5 μM, about 10 μM, about 25 μM, about 50 μM, about 100 μM, or about 1 mM. In some embodiments, the initial amount of a spike-in that is added to the sample is measured by the number of molecules (e.g., about 1, about 10, about 100, about 1000, about 10,000 (10⁴), about 10⁵, about 10⁶, about 10⁷, about 10⁸, about 10⁹, or about 10¹⁰ molecules). In some embodiments, a subsequent measurement of the amount of the spike-in is determined by measuring the molecular count of the spike-in in the extracted DNA sample.

As used herein, the term “corresponds to a target locus” means that the spike-in includes a sequence that is complementary to the sequence of a target locus. For example, “corresponds to a target locus” means that the spike-in includes a sequence that is at least about 80%, at least about 85%, at least about 90%, at least about 95%, at least about 96%, at least about 97%, at least about 98%, or at least about 99% identical to the sequence of a target locus.

In some embodiments, the spike-in is least about 10, 20, 30, 40, 50, 60, 70, 80, 90, 100, 150, 200, 250, 300, 350 nucleotides or more in length.

In some embodiments, the step of adding one or more spike-ins includes adding a dilution series of the spike in or titrating the spike in. In some embodiments, the concentration of the spike in is reduced by about 2-fold, about 5-fold, about 10-fold or about 20-fold at each dilution.

In some embodiments, spike-in includes a concentration ladder of one or more spike-ins for evaluating differential amplification of multiple target loci based on concentration. For example, in some embodiments, a set of spike-ins comprising 2, 3, 4, 5, 6, 7, 8, 9, 10 or more different concentrations of a particular cell or organism or synthetic nucleic acid sequence is added to the sample. In some embodiments, a plurality of sets of spike-ins comprising a concentration ladder are added to the sample (e.g., 2, 3, 4, 5, 6, 7, 8, 9, 10, 11, 12, 13, 14, 15, 16, 17, 18, 19, 20 or more sets of concentration ladders).

In some embodiments, the spike-ins include a set of synthetic nucleic acid composition ladders of varying nucleotide content, wherein the nucleic acid sequences of the spike-ins include a variable sequence region of varying adenine, thymine, cytosine, and/or guanine content flanked by a forward primer binding sequence and a reverse primer binding sequence. In some embodiments, a synthetic nucleic acid spike-in can include a variable sequence region of varying adenine, thymine, cytosine, and/or guanine content (e.g., at least 50%, at least 55%, at least 60%, at least 65%, at least 70%, at least 75%, at least 80%, at least 85%, at least 90%, at least 95%, or more of the variable sequence region is one of adenine, thymine, cytosine, or guanine) flanked by a forward primer binding sequence and a reverse primer binding sequence. In some embodiments, the spike-ins include a set of synthetic nucleic acid composition ladders of varying cytosine and guanine content. Nucleic acid composition ladders can be advantageous, for example, for evaluating biases in the efficiency of nucleic acid capture, enrichment, or amplification based on nucleotide content of the sample or target locus.

In some embodiments, the quantified amounts of spike-ins that are detected in the extracted nucleic acid sample are compared to the initial known amount of the spike-ins that were present in the sample for normalizing the detected amount of the target loci and/or for determining the absolute amount of the target loci. For example, in some embodiments, the measurements obtained from the spike-ins are used to correct for biases in nucleic acid extraction, amplification, and/or sequencing, such as biases with regard to concentration of the target loci, nucleic acid composition (e.g., GC content), and differential relative efficiency of capture or amplification between target loci. In other embodiments, the measurements obtained from spike-ins corresponding to target loci are used to quantify the absolute amounts of the corresponding target loci in the environmental sample.

In some embodiments, the relative efficiency of amplification of a first target locus, relative to a second target locus in the extracted DNA sample is determined. Spike-ins comprising synthetic nucleic acid sequences that correspond to the target loci may be added to the extracted DNA sample. After the amplification reaction, the spike-ins are detected and quantitated. By comparing the quantitated amount of a first spike-in, which corresponds to a first target locus (i.e., includes the primer binding sequences for binding the primers that target a nucleotide sequence of the first target locus) to the quantitated amount of a second spike-in, which corresponds to a second target locus, differences in the efficiency of amplification between the two targets can be determined.

TABLE 1
Exemplary universal backbone sequence. UMI
sequences are represented by “NNNNN”, where “N”
is a degenerate base. Although UMI sequences
containing five “N” degenerate bases are shown,
in some embodiments UMI sequences are between
5 to 20 degenerate bases.

| Description | Sequence | SEQ ID NO |
|---|---|---|
| Universal | NNNNNCCGAGCCCACGAGACGTACGCTGAG | 1 |
| backbone 1 | GGCGGAAAAAATCGTCGGGGACATTGTAAA | |
| | GGCGGCGAGCGCGGCTTTTCCGCGCCAGCG | |
| | TGAAAGCAGTGTGGACTGGCCGTCAGGTAC | |
| | TCGTCGGCAGCGTCNNNNN | |

```
TABLE 2
Exemplary MIP sequences. V refers to the hypervariable region
number; ZZ refers to the backbone sequence (Stefan et al., (2018)
  Sci Reports 8:2028). /5Phos/ indicates that the 5′ end of the
sequence is phosphorylated. The lower-case sequences correspond to
  target locus primers, where the mixed-base code is available at
  the web site www[dot]idtdna[dot]com/pages/products/custom-dna-
rna/mixed-bases. UMI sequences are represented by “NNNNN”,
where “N” is a degenerate base. Although UMI sequences containing
  five “N” degenerate bases are shown, in some embodiments
  UMI sequences are between 5 to 20 degenerate bases.
```

| Description | Sequence | SEQ ID NO |
|---|---|---|
| V2_ZZ | ttactcacccgtycgccrcNNNNNCCGAGCCCACGAGACGTA | 2 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNctgctgcctcccgtaggag | |
| V1V2_ZZ | ctgagccakgatcaaactctNNNNNCCGAGCCCACGAGACGTA | 3 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNctgctgcctcccgtaggag | |
| V3A_ZZ | ctgctgcctcccgtaggagNNNNNCCGAGCCCACGAGACGTA | 4 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNgtattaccgcrgctgctgg | |
| V3B_ZZ | gctgcctcccgtaggagtNNNNNCCGAGCCCACGAGACGTAC | 5 |
| /5Phos/ | GCTGAGGGCGGAAAAAATCGTCGGGGACATTGTAA | |
| | AGGCGGCGAGCGCGGCTTTTCCGCGCCAGCGTGAA | |
| | AGCAGTGTGGACTGGCCGTCAGGTACTCGTCGGCA | |
| | GCGTCNNNNNgtattaccgcrgctgctgg | |
| V6V7A_ZZ | ggtaaggttyytcgcgttgcNNNNNCCGAGCCCACGAGACGTA | 6 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNgacgtcrtccccaccttcc | |
| V6V7B_ZZ | ggtaaggttyytcgcgttgcNNNNNCCGAGCCCACGAGACGTA | 7 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNttgacgtcrtccccaccttcc | |
| V6A_ZZ | gcgggbccccgtcaattcNNNNNCCGAGCCCACGAGACGTA | 8 |
| /5Phos/ | CGCTGAGGGCGGAAAAAATCGTCGGGGACATTGTA | |
| | AAGGCGGCGAGCGCGGCTTTTCCGCGCCAGCGTGA | |
| | AAGCAGTGTGGACTGGCCGTCAGGTACTCGTCGGC | |
| | AGCGTCNNNNNgagctgacgacarccatgc | |
| V6B_ZZ | ccccgtcaattcmtttragtttNNNNNCCGAGCCCACGAGACGT | 9 |
| /5Phos/ | ACGCTGAGGGCGGAAAAAATCGTCGGGGACATTGT | |
| | AAAGGCGGCGAGCGCGGCTTTTCCGCGCCAGCGTG | |
| | AAAGCAGTGTGGACTGGCCGTCAGGTACTCGTCGG | |
| | CAGCGTCNNNNNagggttgcgctcgttg | |

TABLE 3
Exemplary sequencing primer sequences. [i7]
and [i5] indicate index sequences. Additional
portions of adapter sequences are contained
in MIP universal backbone sequences.

| Description | Sequence | SEQ ID NO |
|---|---|---|
| P7 Primer | CAAGCAGAAGACGGCATACGAGAT | The |
| | [i7]GTCTCGTGGGCTCGG | underlined |
| | | sequence |
| | | corresponds |
| | | to SEQ ID |
| | | NO: 10; and |
| | | the bolded |
| | | sequence |
| | | corresponds |
| | | to SEQ ID |
| | | NO: 11. |
| P5 Primer | AATGATACGGCGACCACCGAGATCT | The |
| | ACAC[i5]TCGTCGGCAGCGTC | underlined |
| | | sequence |
| | | corresponds |
| | | to SEQ ID |
| | | NO: 29; and |
| | | the bolded |
| | | sequence |
| | | corresponds |
| | | to SEQ ID |
| | | NO: 30. |

TABLE 4
Exemplary sequencing primer sequences including
index sequences. Additional portions of adapter
sequences are contained in MIP universal backbone
sequences

| | | SEQ |
|---|---|---|
| Description | Sequence | ID NO |
| P7 Primer-1 | CAAGCAGAAGACGGCATACGAGATGTGAAT | 12 |
| | ATGTCTCGTGGGCTCGG | |
| P7 Primer-2 | CAAGCAGAAGACGGCATACGAGATACAGGC | 13 |
| | GCGTCTCGTGGGCTCGG | |
| P7 Primer-3 | CAAGCAGAAGACGGCATACGAGATCATAGA | 14 |
| | GTGTCTCGTGGGCTCGG | |
| P7 Primer-4 | CAAGCAGAAGACGGCATACGAGATTGCGAG | 15 |
| | ACGTCTCGTGGGCTCGG | |
| P7 Primer-5 | CAAGCAGAAGACGGCATACGAGATTCTCTA | 16 |
| | CTGTCTCGTGGGCTCGG | |
| P7 Primer-6 | CAAGCAGAAGACGGCATACGAGATCTCTCG | 17 |
| | TCGTCTCGTGGGCTCGG | |
| P7 Primer-7 | CAAGCAGAAGACGGCATACGAGATCCAAGT | 18 |
| | CTGTCTCGTGGGCTCGG | |
| P7 Primer-8 | CAAGCAGAAGACGGCATACGAGATTTGGAC | 19 |
| | TCGTCTCGTGGGCTCGG | |
| P7 Primer-9 | CAAGCAGAAGACGGCATACGAGATGCAGAA | 20 |
| | TTGTCTCGTGGGCTCGG | |
| P7 Primer-10 | CAAGCAGAAGACGGCATACGAGATATGAGG | 21 |
| | CCGTCTCGTGGGCTCGG | |
| P7 Primer-11 | CAAGCAGAAGACGGCATACGAGATACTAAG | 22 |
| | ATGTCTCGTGGGCTCGG | |
| P7 Primer-12 | CAAGCAGAAGACGGCATACGAGATGTCGGA | 23 |
| | GCGTCTCGTGGGCTCGG | |
| P7 Primer-13 | CAAGCAGAAGACGGCATACGAGATAGCCTC | 24 |
| | ATGTCTCGTGGGCTCGG | |
| P7 Primer-14 | CAAGCAGAAGACGGCATACGAGATGATTCT | 25 |
| | GCGTCTCGTGGGCTCGG | |
| P7 Primer-15 | CAAGCAGAAGACGGCATACGAGATTCGTAG | 26 |
| | TGGTCTCGTGGGCTCGG | |
| P7 Primer-16 | CAAGCAGAAGACGGCATACGAGATCTACGA | 27 |
| | CAGTCTCGTGGGCTCGG | |
| P5 Primer-1 | AATGATACGGCGACCACCGAGATCTACACA | 28 |
| | GCGCTAGTCGTCGGCAGCGTC | |

### EXAMPLES

### Example 1: Design of Molecular Inversion Probes with Unique Molecular Identifiers for Quantitative Microbial Community Profiling

In this Example, a method for designing molecular inversion probes (MIPs) for quantitative microbial community profiling is provided.

Design of MIPs for Quantitative Microbial Community Profiling

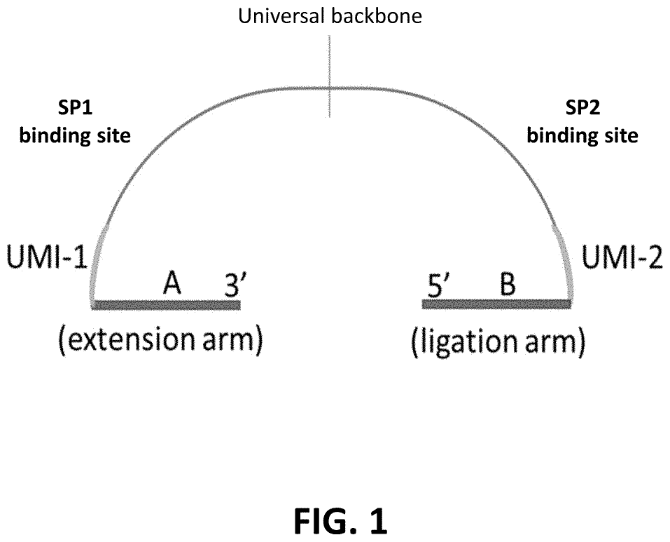

As shown in FIG. 1, MIPs for quantitative microbial community profiling include two target locus primers, “A” and “B”, which are also termed “extension arm” and “ligation arm”, respectively. The target locus primers are complementary to non-overlapping sequences that flank a target locus. For example, the target locus may be a 16S/18S ribosomal subunit gene, an internal transcribed spaces (ITS) region, a microbial sequence (e.g. a special or strain identifier), a target locus that distinguishes a pathogenic microorganism from a non-pathogenic or beneficial microorganism, and/or a functional gene associated with particular pathway and/or biochemical processes (e.g., nifDHK for nitrogen fixation, amoABC for ammonia oxidation).

The target locus primers are connected by a universal backbone sequence. Adjacent to each target locus primer is a unique molecular identifier sequence consisting of between 5 and 20 or more degenerate nucleotides (“UMI-1”, “UMI-2”, “UMI-3”, etc.). The universal backbone sequence also contains one sequencing primer binding site adjacent to each UMI (“SP1” and “SP2”).

### Example 2: Quantitative Microbial Community Profiling

Hybridization of MIPs to Target Nucleic Acid Molecules and Extension Ligation

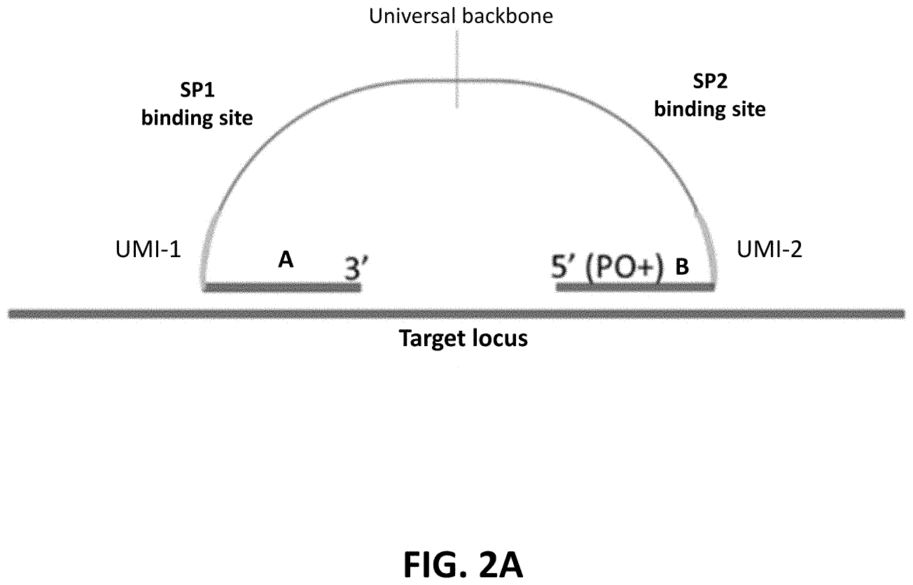

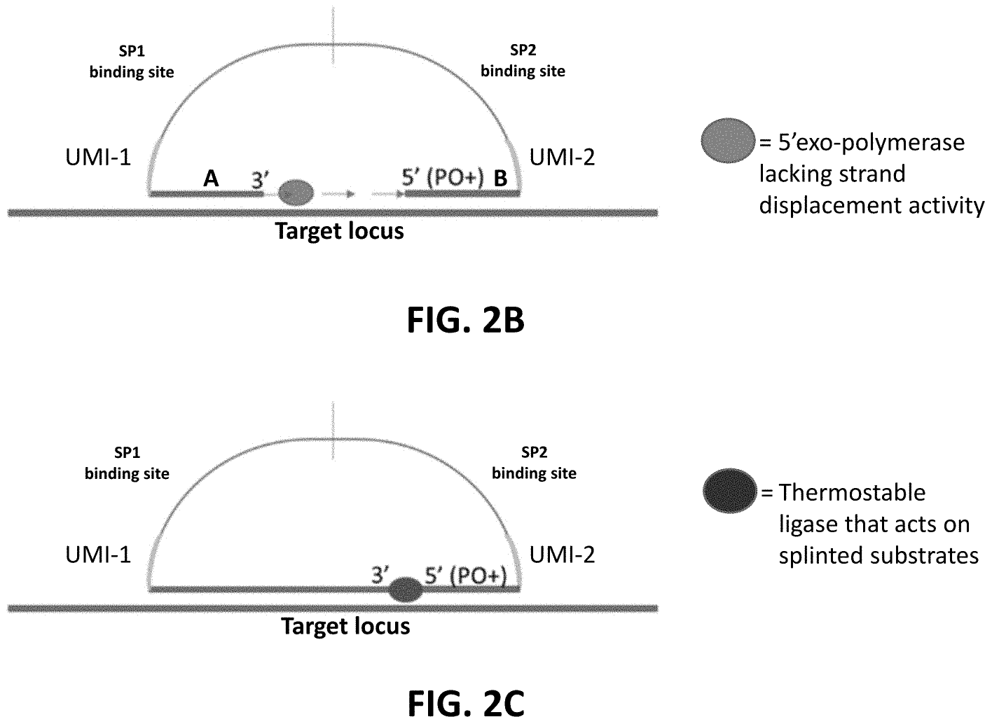

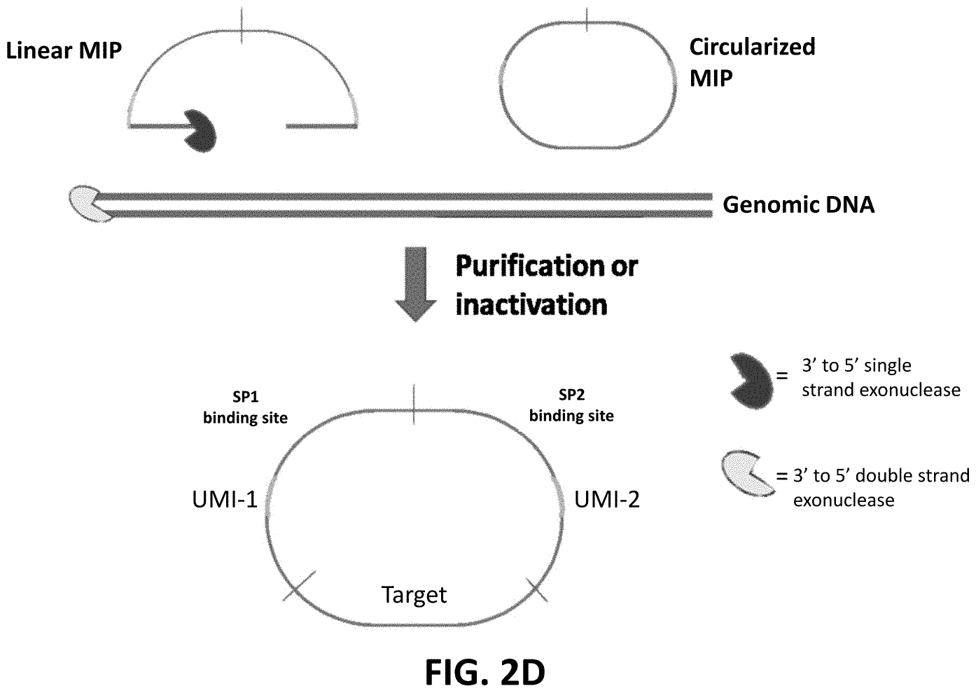

Sample nucleic acids are denatured by incubating at 98° C. in the presence of the desired MIP or pool of MIPs. The sample is then ramped down to a temperature of between 50° C. to 55° C. to allow hybridization of the MIP to its corresponding target molecules. As depicted in FIG. 2A, when both target locus primers bind their complementary sequences flanking the target locus in the sample nucleic acids, the MIP circularizes.

An extension and ligation reaction is performed using a reaction mixture containing: dNTPS, appropriate buffers (e.g., 2× Phusion master mix), a 5′exo-polymerase lacking strand displacement activity, such as Stoffel fragment, TaqIT, Klenow Large Fragment, or Phusion polymerase, and a thermostable ligase that acts on splinted substrates, such as Ampligase. The reactions are incubated at a temperature of between 50° C. to 55° C. for 30 minutes to 90 minutes. During the incubation, the 5′exo-polymerase lacking strand displacement activity synthesizes a sequence complementary to the target locus sequence starting at the 3′ end of target locus primer “A” (extension arm) until reaching target locus primer “B” (ligation arm) at the 5′-phosphorylated end of the MIP (FIG. 2B). The thermostable ligase that acts on splinted substrates to ligate the 3′ end of the newly synthesized nucleic acid fragment to the 5′-phosphorylated end of the MIP, thereby generating an uninterrupted circular molecule (FIG. 2C).

Following extension and ligation, a mixture containing two exonucleases is employed to eliminate unused MIPs and nucleic acid templates (FIG. 2D): a 3′ to 5′ single strand exonuclease, such as exonuclease I, is used to degrade linear single-stranded substrates (e.g., unused MIPs, denatured genomic DNA, denatured cDNA; a 3′ to 5′ double strand exonuclease, such as exonuclease III, is used to degrade linear double stranded substrates (genomic DNA, and spurious heterodimers formed during extension and ligation steps). Following degradation, exonucleases are removed by heat inactivation and/or purification of nucleic

Amplification and Sequencing Library Preparation

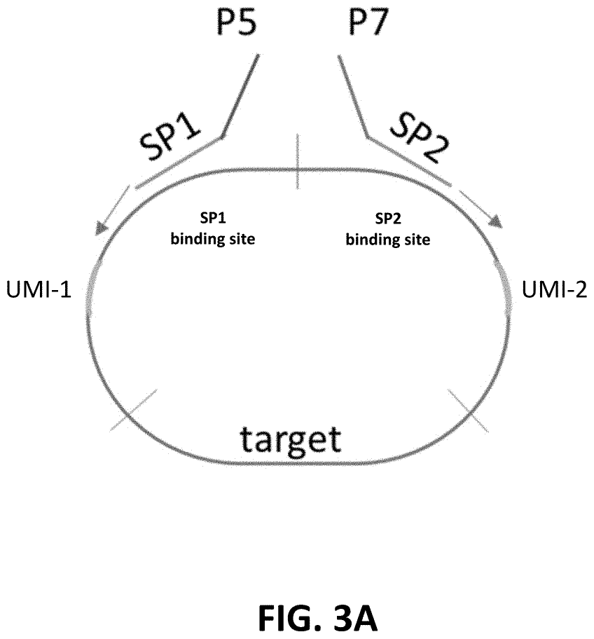

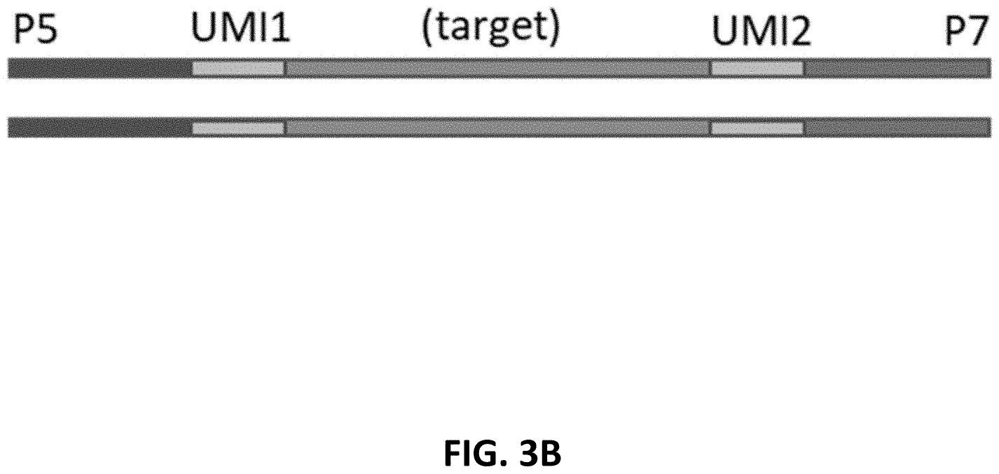

An aliquot of the resulting circular templates is then used as input into a polymerase chain reaction (PCR) to amplify and prepare the sample library for sequencing. The PCR reaction mixture includes one forward primer and one reverse primer that bind to the sequencing primer binding sites on the universal backbone (SP1 and SP2). As shown in FIG. 3A, each of the primers also contain a sequencing adapter “primer tail” (P5 and P7 sequences) that is required for fragment recognition by the sequencer. In addition, the primers contain sample index sequences that correspond to a specific sample, thus enabling nucleic acids from multiple samples to be sequenced simultaneously.

The PCR amplification reactions generate linear double stranded DNA products that contain a sequencing adapter sequence on each end (P5 and P7), sample index sequence(s) and a target sequence flanked by two UMI sequences (FIG. 3B). Following completion of the amplification reactions, the reaction products are purified, quantified, and their concentrations are normalized prior to being pooled for sequencing.

A summary of the conditions for hybridization of MIPs to target nucleic acid molecules and Extension Ligation, removal of unused MIPs and nucleic acid templates, and amplification and sequencing library preparation is provided in Table 5.

TABLE 5
Conditions for hybridization of MIPs to target nucleic
acid molecules and Extension Ligation, removal of
unused MIPs and nucleic acid templates, and amplification
and sequencing library preparation.

| Hybridization and Ligation | Stock | Vol Added (μl) |
|---|---|---|
| 2X Phusion Mastermix | 2 | 7.5 |
| Ampligase (units/μl) | 100 | 0.15 |
| NAD (mM) Dilute First | 5 | 1.5 |
| Probes (total nM) | 100 | 1 |
| Sample (10 ng) | NA | 5 |

| | |
|---|---|
| Total Reaction Volume | 15.15 |

Reaction: 98° C. for 3 minutes, 4 hrs at 55° C.,
15 minutes at 72° C., 4° C. forever.
Exo

| | | |
|---|---|---|
| ExoI (units/μl) | 100 | 1.0 |
| ExoIII (units/μl) | 100 | 0.3 |

| | |
|---|---|
| Total Volume added | 1.3 |
| Total Reaction Volume | 16.45 |

Reaction: 37° C. for 30 min, 80° C. for 20 min, hold at 4° C.
PCR

| | | |
|---|---|---|
| 1X Phusion Mastermix | 1X | 25 |
| (diluted in HF buffer) | | |
| Fwd Primer (uM) | 10 | 2.5 |
| Water | | 3.6 |

| | |
|---|---|
| Total Volume Added | 31.1 |

98° C. C. f/3 min, 30 cycles: 98° C. for 10 s, 60° C. for 30 s,
72° C. for 15 sec, 72° C. for 5 min, 4° C. hold.

| | | |
|---|---|---|
| Reverse Indexing Primer (uM) | 10 | 2.5 |

| | |
|---|---|
| Total Reaction Volume | 50.05 |

Purify using 0.7X Ampure
1X 150 μl Wash with 80% Ethanol, elute into 25 μl
Qiagen elution buffer (10 mM Tris-HCl).

Sequencing

Sequencing of the samples is performed with massively parallel sequencing using reversible chain termination, such as with the Illumina sequencing platform.

Demultiplexing of Sequencing Data (Grouping) by Sample Index

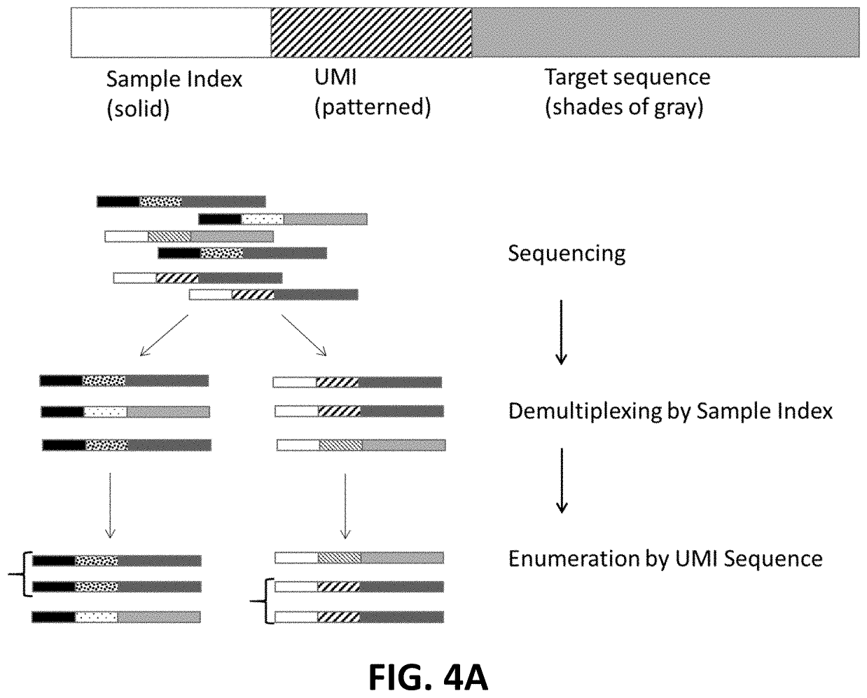

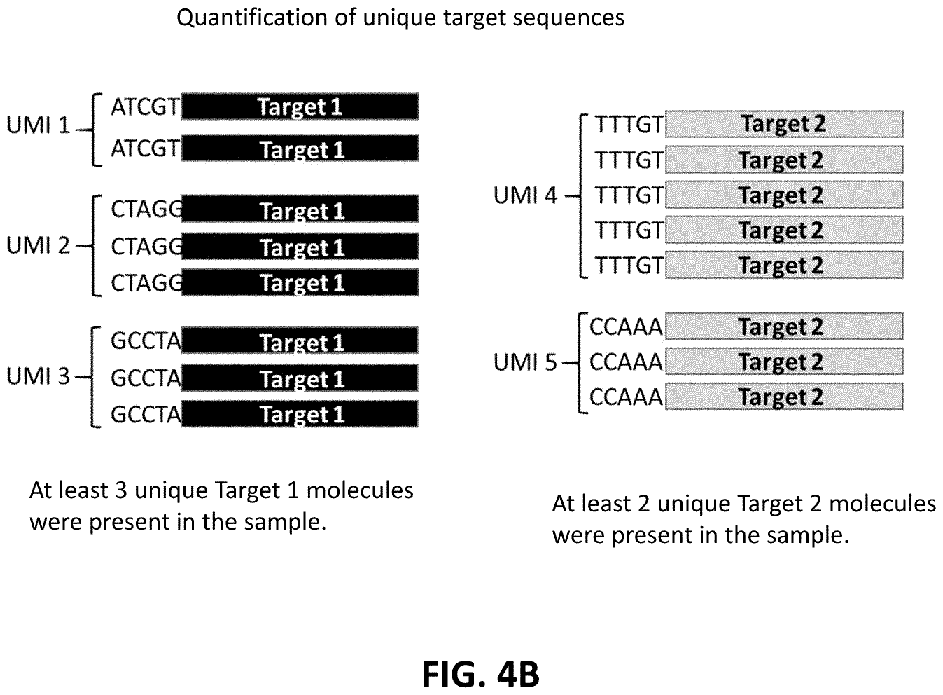

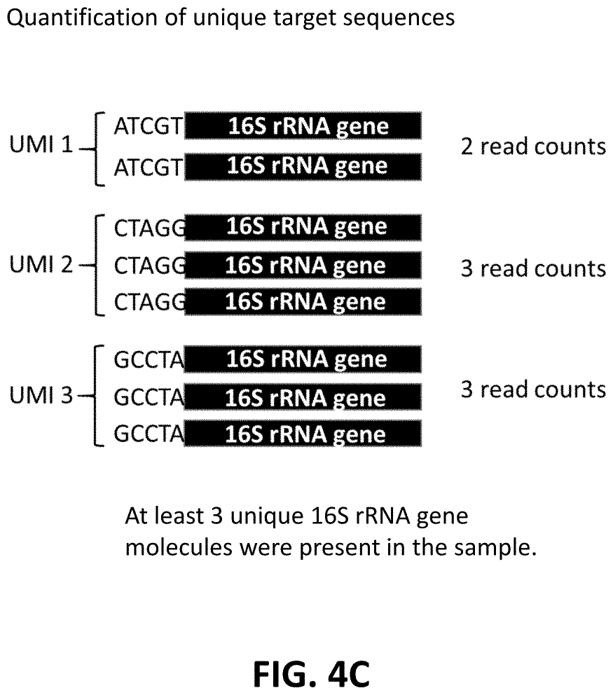

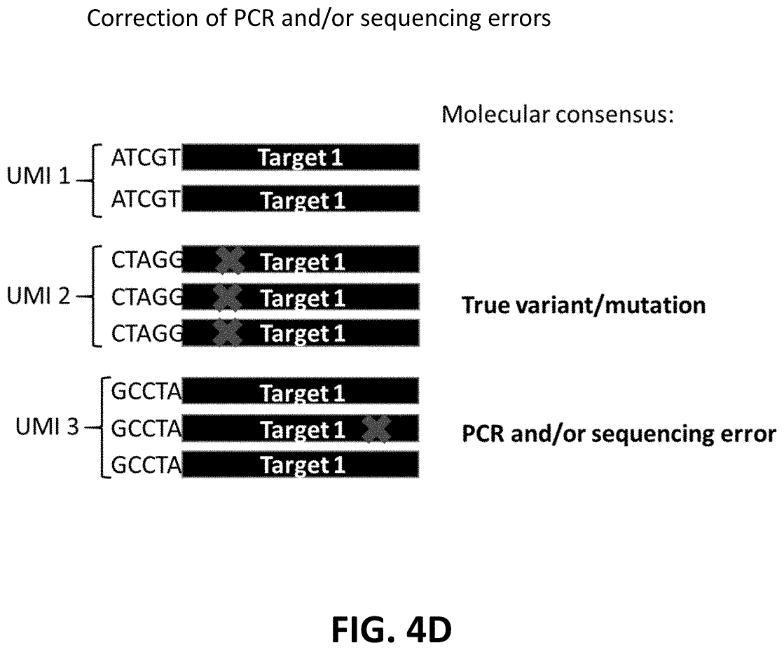

After sequencing, the resulting plurality of sequencing reads are demultiplexed by binning the reads according the sample index sequences, such that all reads carrying the same index sequence are grouped (FIG. 4A).

Enumeration of Sequencing Data by UMI Sequence and Quantification of Original Progenitor Molecules

As shown in FIG. 4A, each group of sequencing reads corresponding to a single sample index sequence is then further demultiplexed by binning the reads according to the UMI sequence, such that all reads carrying the same UMI sequence are grouped.

To quantify the number of unique target locus progenitor molecules that were present in a nucleic acid sample prior to amplification, as shown in FIGS. 4A-4B, for each unique target locus sequence, the number of unique UMIs associated with all reads with the identical target locus sequence are counted in order to obtain the number of unique target locus progenitor molecules of the target locus sequence that were present in the sample prior to amplification. For example, if a given sequence has a read count of 32 with three different observed UMI sequences, the corresponding 32 reads are collapsed into three unique reads, indicating that at least three original progenitor molecules corresponding to that target sequence were present in the original sample.

In one example, as shown in FIG. 4C, the sequencing data shows two counts of the 16S rRNA gene sequence with UMI 1, three counts with UMI 2, and three counts with UMI 3. In this example, the two counts of the sequence with UMI 1 are duplicates, as are the three counts with UMI 2 and the three counts with UMI3, therefore there were three original progenitor molecules corresponding to the 16S rRNA gene in the sample.

The quantification of unique original progenitor molecules is then translated into cell counts, microbial abundance, gene counts, or chemical availability and/or Transformation Process Rates in the original sample, as described in Example 3 and Example 4 below.

Error Correction

Multiple reads corresponding to an original progenitor molecule (i.e., the same target sequence and UMI sequence) in a sample are then used to correct sequencing (e.g., base calling errors) and/or PCR errors (e.g., polymerase errors) by forming a molecular consensus across the reads. For example, as shown in FIG. 4D, a sequence variation (e.g., base substitution) that appears only in a subset of sequencing reads corresponding to a single original progenitor molecule (i.e., sequencing reads with identical UMI sequences) is likely to be due to a PCR or sequencing error. In contrast, a sequence variation that appears in all sequencing reads corresponding to a single original progenitor molecule is likely to be a true sequence variant that was present in the original nucleic acid sample.

Alignment

Sequencing reads are aligned to a collection of reference sequences, such as annotated reference genomes, to infer functional or phylogenetic information. Alignments are carried using translated query searches and nucleotide-based query searches.

### Example 3: Quantification of Microbial Abundances

In this Example, MIPs as described in Example 1 are used to quantify microbial taxa within a soil sample.

MIPs targeting 16S rRNA are used to quantify bacterial taxa, while the MIPs targeting the ITS2 gene or 18S rRNA are used to quantify fungal taxa.

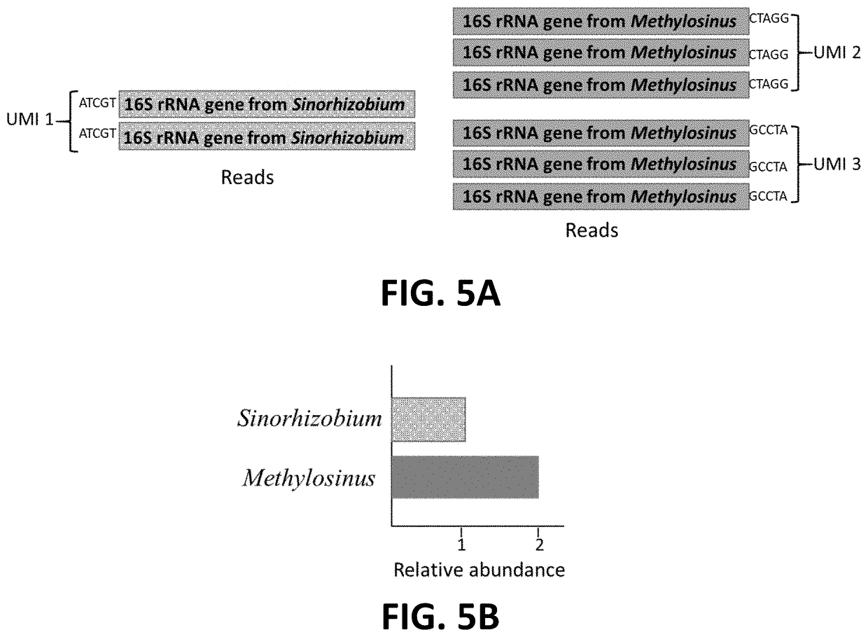

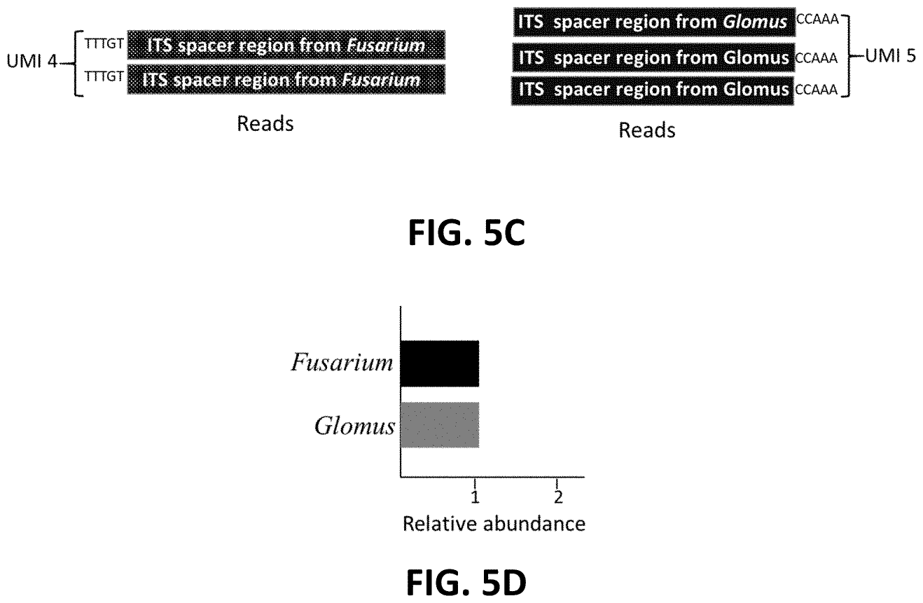

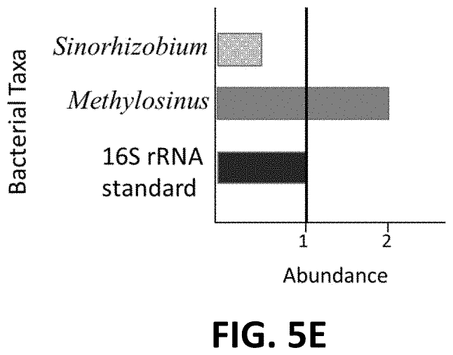

In one example, soil DNA extracts are denatured and hybridized to MIPs targeting the 16S rRNA gene to quantify the relative abundance of bacterial taxa in a sample. The samples are processed, sequenced and demultiplexed as described in Example 2. As shown in FIG. 5A, alignment of sequencing data shows that two reads with UMI 1 map to a 16S rRNA sequence belonging to the Sinorhizobium genus of rhizobial nitrogen-fixing bacteria, while three reads with each one of UMI 2 and UMI 3 map to a 16S rRNA sequence belonging to the Methylosinus genus of methanotrophic nitrogen-fixing bacteria. Accordingly, the numbers of unique progenitor molecules mapping to Sinorhizobium and Methylosinus are 1 and 2, respectively. Thus, as shown in FIG. 5B, the respective relative abundance of the Sinorhizobium and Methylosinus genera in the sample is 1:2.

In another example, soil DNA extracts are denatured and hybridized to MIPs targeting the ITS spacer region to quantify the relative abundance of fungal taxa in a sample. The samples are processed, sequenced, and demultiplexed as described in Example 2. As shown in FIG. 5C, alignment data shows that two reads with UMI 4 map to ITS spacer region of the fungal pathogen Fusarium, while three reads with UMI 5 map to the ITS spacer region of the fungal symbiont Glomus. Accordingly, the number of unique progenitor molecules mapping to Fusarium and Glomus is 1 for both taxa. Thus, as depicted in FIG. 5D, the relative abundance of Fusarium to Glomus in the sample is 1:1.

In another example, multiple microbial taxa are quantified simultaneously when a soil DNA extract is combined with multiple MIPs targeting different taxa, for example, bacterial and fungal taxa.

To determine the absolute abundance of microbial taxa within a sample, known amounts of targets in varying proportions are added to or titrated into the sample. For example, known amounts of standards such as different mixtures of bacterial cells and/or fungal cells corresponding to the taxa of interest are added (spiked-in) to a soil sample. Alternatively, known amounts of DNA, including DNA constructs (e.g., synthetic DNA) corresponding to target loci of interest are spiked into a DNA extract from a soil sample. Following sequencing and quantification as described in Examples 1 and 2, the relative rates of amplification for each target are determined from the known amounts of the standards (spike-ins), and the relative abundances of the targets are compared to the abundances of their corresponding standards.

In one example, a known amount of the 16S rRNA locus standard is spiked into a DNA extract from a soil sample containing bacterial cells of the Sinorhizobium and Methylosinus taxa. As shown in FIG. 5E, following sequencing and quantification as described in Examples 1 and 2, the absolute abundances of Sinorhizobium and Methylosinus are determined by comparing the measured abundance of the 16S rRNA locus for each species to the measured abundance and known spike-in amount of 16S rRNA locus standard.

Using the approaches described in this Example, both the overall relative and absolute abundances of all microbial organisms present in a sample are determined. In addition, the data is used to focus further analysis of the soil sample on the abundance of individual species, such as specific plant pathogens of interest for a specific crop.

### Example 4: Determination of Chemical Availability and Transformation Process Rates

In addition to determining taxonomic features of a soil microbiome, as shown in this Example, the MIPs described herein are also used to quantify functional genes and chemical availability and/or Transformation Process Rates in a soil sample.

For example, the relative and/or absolute abundances in a soil sample of genes that function in metabolic pathways, or biological pathways related to element cycling in soil, transforming forms of nitrogen, phosphorous, carbon, or oxygen, or plant growth promotion are quantified. Chemical availability and/or Transformation Process Rates in the soil sample are then determined based on the abundances of genes in the soil sample.

In one example, the abundances of the gene nifH, which functions in nitrogen fixation, and the gene nirK, which functions in denitrification, are quantified. Soil DNA extracts are combined with MIPs targeting the nifH or the nirK genes. The samples are then processed, sequenced, demultiplexed, and quantified as described in Example 2.

As described in the preceding Examples, UMIs for each target locus (nifH or nirK) are used to count unique progenitor molecules corresponding to each target locus that were present in the soil sample. UMIs are also used for error correction and/or variant detection. An external standard (e.g. synthetic DNA construct) corresponding to each target locus is used to determine the absolute abundances of these targets, given calibration assays to determine the relative preference for each target during amplification, hence giving a total abundance (or ratio) of nifH and nirK in a given soil DNA extract. The availability and/or Transformation Process Rate of nitrogen in the soil is then determined based on the abundances of nifH and nirK in the soil sample.

In addition, redundant MIPs targeting each target locus are used to count occurrences of different organisms which carry the target loci by aligning to reference genomes (as described above), and therefore determining phylogenetic origin of the particular target locus sequence. In one example, the relative (or absolute) abundance of different organisms that carry nifH genes are determined by identifying the organism from which each specific gene sequence came from, and comparing the nifH counts of unique progenitor molecules calculated by UMIs. This information is of considerable agricultural and ecological interest if, for example, one such organism is a crop-specific symbiotic nitrogen fixer (e.g., Sinorhizobium fredii), while another is a free-living nitrogen fixer dependent on methane availability (e.g., Methlyosinus trichosporium).

1. A method for profiling of microorganisms in an environmental sample, wherein the method comprises
a) extracting DNA from the environmental sample;
b) denaturing the extracted DNA;
c) incubating the denatured DNA with a molecular inversion probe (MIP) under conditions that allow hybridization,
wherein the MIP comprises
(i) in the 3′ to 5′ direction,
a first target locus primer, wherein the first primer comprises a nucleotide sequence complementary to a first sequence in a target locus,
a universal backbone sequence comprising a first sequencing primer binding site and a second sequencing primer binding site, and
a second target locus primer, wherein the second primer comprises a nucleotide sequence complementary to a second, non-overlapping sequence in the target locus, and
(ii) a first unique molecular identifier (UMI);
wherein the backbone sequence has low sequence homology to DNA in the environmental sample and has minimal ability to form secondary structures,
thereby generating a sample comprising denatured DNA-MIP complexes;
d) after hybridization, performing an extension and ligation reaction comprising incubating the sample comprising denatured DNA-MIP complexes with nucleotides, 5′ exo-polymerase lacking strand displacement activity, and a thermostable ligase capable of ligating splinted substrates under conditions that allow extension of the 3′ end of the MIP and ligation to the 5′ end of the MIP;
e) after extension and ligation, incubating the sample comprising denatured DNA-MIP complexes with a 3′ to 5′ single strand exonuclease and a 3′ to 5′ double strand exonuclease under conditions sufficient to degrade linear substrates, thereby generating a sample comprising circular DNA templates;
f) removing the 3′ to 5′ single strand exonuclease and the 3′ to 5′ double strand exonuclease from the sample comprising circular DNA templates;
g) amplifying the circular DNA templates, thereby generating linear DNA comprising the sequence of the MIP from the 5′ end of the first primer binding site to the 3′ end of the second primer binding site; and
h) sequencing the linear DNA, thereby generating a plurality of sequencing reads comprising the sequence of the linear DNA, thereby profiling the microorganisms in the environmental sample.

2. The method of claim 1, wherein the first UMI is between the first target locus primer and the first sequencing primer binding site.

3. The method of claim 1, wherein the MIP further comprises a second UMI.

4. The method of claim 3, wherein the second UMI is between the second target locus primer and the second sequencing primer binding site.

5. The method of claim 1 wherein the first UMI and the second UMI each comprise between 5 and 20 bases.

6. The method of claim 1, wherein the first target locus primer and the second target locus primer comprise at least one degenerate nucleotide base at the 3′ end and/or the 5′ end.

7. The method of claim 1, wherein the denatured DNA is incubated with a second MIP comprising a first and a second target locus primer complementary to sequences in a second target locus.

8. The method of claim 1, wherein the 5′ exo-polymerase lacking strand displacement activity is selected from the group consisting of Stoffel fragment, TaqIT, Kienow large fragment, and Phusion polymerase.

9. The method of claim 1, wherein the thermostable ligase capable of ligating splinted substrates is selected from the group consisting of Taq ligase, T4 DNA ligase, and Ampligase.

10. The method of claim 1, wherein the 3′ to 5′ single strand exonuclease is exonuclease I.

11. The method of claim 1, wherein the 3′ to 5′ double strand exonuclease is exonuclease III or Kamchatka crab nuclease.

12. The method of claim 1, wherein the 3′ to 5′ single strand exonuclease and the 3′ to 5′ double strand exonuclease are removed by heat inactivation and/or purification.

13. The method of claim 1, wherein the step of amplifying comprises polymerase chain reaction (PCR) comprising a PCR reaction mix, wherein the PCR reaction mix comprises a high-fidelity proof-reading polymerase and sequencing primers.

14. The method of claim 13, wherein the sequencing primers comprise:
a sequence complementary to the first or second sequencing primer binding sites, and a P5 or a P7 sequence.

15. The method of claim 14, wherein the sequencing primers further comprise a sample index.

16. The method of claim 1, wherein the sequencing comprises sequencing with massively parallel sequencing using reversible chain termination.

17. The method of claim 1, wherein the sequencing reads are paired-end reads.

18. The method of claim 1, further comprising grouping sequencing reads if they comprise the same sample index sequence, thereby generating bins comprising sequencing reads from the same sample.

19. The method of claim 1, further comprising grouping the sequencing reads from the same sample if they comprise the same UMI sequence, thereby generating bins comprising sequencing reads from the same sample and with the same UMI sequence, thereby quantifying the number of unique target loci in the sample.

20. The method of claim 19, further comprising analyzing the sequencing reads from the same sample and with the same UMI sequence to determine whether the sequencing reads have the same or a different nucleotide at each position.

21. The method of claim 1, further comprising aligning the sequencing reads to a collection of reference sequences, thereby identifying the microorganisms in the environmental sample.

22. The method of claim 21, further comprising analyzing the sequencing reads from the same sample and target locus to determine whether the sequencing reads have the same or a different nucleotide at each position relative to the reference sequence.

23. The method of claim 1, further comprising determining microbial abundance in the environmental sample based on the number of unique target loci in the environmental sample.

24. The method of claim 1, further comprising determining chemical availability and/or Transformation Process Rates in the environmental sample based on the number of unique target loci and/or the microorganisms identified in the environmental sample.

25. The method of claim 1, wherein a known amount of a spike-in is added to the environmental sample prior to the step of extracting DNA from the environmental sample, and wherein the spike-in is selected from the group consisting of bacterial cells, fungal cells, viral particles, and any combinations thereof.

26. The method of claim 1, wherein a known amount of a spike-in is added to the extracted DNA, and wherein the spike-in is selected from the group consisting of DNA constructs, synthetic DNA, DNA fragments, and any combinations thereof.

27. The method of claim 1, wherein the target locus is a taxonomic marker selected from the group consisting of a 16S ribosomal RNA, an 18S ribosomal RNA, an internal transcribed spacer (ITS) region, a microbial sequence that identifies a species and/or strain, and a target locus that distinguishes a pathogenic microorganism from a non-pathogenic and/or beneficial microorganism.

28. The method of claim 1, wherein the target locus is a gene associated with a biological pathway selected from the group consisting of:
cycling or transformation of compounds containing nitrogen, nitrogen fixation, ammonia oxidation, nitrification, denitrification, organic nitrogen mineralization, mineral nitrogen immobilization, organic nitrogen immobilization,
cycling or transformation of compounds containing phosphorous, mineral phosphorous solubilization, hydrolysis of organic phosphorous compounds, hydrolysis of inorganic phosphorous polymers, immobilization of phosphorous,
cycling or transformation of compounds containing carbon, uptake or degradation of sugars, uptake or degradation of oligosaccharides, uptake or degradation of polysaccharides, uptake or degradation of structural polymers,
uptake or degradation of cellulose, uptake or degradation of hemicellulose, uptake or degradation of lignocellulose, uptake or degradation of lignin,
uptake or degradation of aliphatic compounds, uptake or degradation of alkane compounds, uptake or degradation of aromatic compounds,
metabolic pathways for aerobic respiration, metabolic pathways for anaerobic respiration, aerobic cytochrome oxidation, microaerobic cytochrome oxidation, anaerobic respiration utilizing nitrate, iron, manganese, sulfate, acetate, or CO₂ as terminal electron acceptors, anaerobic cytochrome oxidation, and any combinations thereof.

29. The method of claim 1, wherein the target locus is a gene associated with a process selected from the group consisting of agricultural processes, plant growth, plant disease, cycling of micronutrients, cycling of potassium, cycling of zinc, cycling of calcium, plant growth promotion, production of indole-3-acetic acid (IAA), production of siderophores, production of 1-amino-cyclopropane-1-carboxylate (ACC) deaminase, production of hydrogen cyanate, nutrition, N-fixation, P solubilization, disease suppression in the soil, antibiotic resistance, and any combinations thereof.

30. The method of claim 1, wherein the environmental sample comprises soil.

31. The method of claim 1, wherein the environmental sample comprises bacterial cells, fungal cells, nematodes, and/or virus particles.

32-41. (canceled)


SEQUENCE LISTING (30 sequences)

SEQ ID NO: 1 - DNA, 139 bp, Artificial Sequence
  Synthetic Construct
  misc_feature 1, 2, 3, 4, 5, 135, 136, 137, 138, 139: n = A,T,C or G
nnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnn

SEQ ID NO: 2 - DNA, 177 bp, Artificial Sequence
  Synthetic Construct
  misc_feature 13: n = C or T
  misc_feature 18: n = A or G
  misc_feature 20, 21, 22, 23, 24, 154, 155, 156, 157, 158: n = A,T,C or G
ttactcacccgtncgccncnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnnctgctgcctcccgtaggag

SEQ ID NO: 3 - DNA, 178 bp, Artificial Sequence
  Synthetic Construct
  misc_feature 9: n = G or T
  misc_feature 21, 22, 23, 24, 25, 155, 156, 157, 158, 159: n = A,T,C or G
ctgagccangatcaaactctnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnnctgctgcctcccgtaggag

SEQ ID NO: 4 - DNA, 177 bp, Artificial Sequence
  Synthetic Construct
  misc_feature 20, 21, 22, 23, 24, 154, 155, 156, 157, 158: n = A,T,C or G
  misc_feature 169: n = A or G
ctgctgcctcccgtaggagnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnngtattaccgcngctgctgg

SEQ ID NO: 5 - DNA, 176 bp, Artificial Sequence
  Synthetic Construct
  misc_feature 19, 20, 21, 22, 23, 153, 154, 155, 156, 157: n = A,T,C or G
  misc_feature 168: n = A or G
gctgcctcccgtaggagtnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnngtattaccgcngctgctgg

SEQ ID NO: 6 - DNA, 178 bp, Artificial Sequence
  Synthetic Construct
  misc_feature 10, 11: n = C or T
  misc_feature 21, 22, 23, 24, 25, 155, 156, 157, 158, 159: n = A,T,C or G
  misc_feature 166: n = A or G
ggtaaggttnntcgcgttgcnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnngacgtcntccccaccttcc

SEQ ID NO: 7 - DNA, 180 bp, Artificial Sequence
  Synthetic Construct
  misc_feature 10, 11: n = C or T
  misc_feature 21, 22, 23, 24, 25, 155, 156, 157, 158, 159: n = A,T,C or G
  misc_feature 168: n = A or G
ggtaaggttnntcgcgttgcnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnnttgacgtcntccccaccttcc

SEQ ID NO: 8 - DNA, 176 bp, Artificial Sequence
  Synthetic Construct
  misc_feature 6: n = T,C or G
  misc_feature 19, 20, 21, 22, 23, 153, 154, 155, 156, 157: n = A,T,C or G
  misc_feature 170: n = A or G
gcgggnccccgtcaattcnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnngagctgacgacanccatgc

SEQ ID NO: 9 - DNA, 177 bp, Artificial Sequence
  Synthetic Construct
  misc_feature 13: n = A or C
  misc_feature 23, 24, 25, 26, 27, 157, 158, 159, 160, 161: n = A,T,C or G
  misc_feature 17: n = A or G
ccccgtcaattcntttnagtttnnnnnccgagcccacgagacgtacgctgagggcggaaaaaatcgtcggggacattgtaaaggcggcgagcgcggcttttccgcgccagcgtgaaagcagtgtggactggccgtcaggtactcgtcggcagcgtcnnnnnagggttgcgctcgttg

SEQ ID NO: 10 - DNA, 24 bp, Artificial Sequence
  Synthetic Construct
  misc_feature 24: Linked to position 1 of SEQ ID NO11 by an index sequence
caagcagaagacggcatacgagat

SEQ ID NO: 11 - DNA, 15 bp, Artificial Sequence
  Synthetic Construct
  misc_feature 1: Linked to position 24 of SEQ ID NO10 by an index sequence
gtctcgtgggctcgg

SEQ ID NO: 12 - DNA, 47 bp, Artificial Sequence
  Synthetic Construct
caagcagaagacggcatacgagatgtgaatatgtctcgtgggctcgg

SEQ ID NO: 13 - DNA, 47 bp, Artificial Sequence
  Synthetic Construct
caagcagaagacggcatacgagatacaggcgcgtctcgtgggctcgg

SEQ ID NO: 14 - DNA, 47 bp, Artificial Sequence
  Synthetic Construct
caagcagaagacggcatacgagatcatagagtgtctcgtgggctcgg

SEQ ID NO: 15 - DNA, 47 bp, Artificial Sequence
  Synthetic Construct
caagcagaagacggcatacgagattgcgagacgtctcgtgggctcgg

SEQ ID NO: 16 - DNA, 47 bp, Artificial Sequence
  Synthetic Construct
caagcagaagacggcatacgagattctctactgtctcgtgggctcgg

SEQ ID NO: 17 - DNA, 47 bp, Artificial Sequence
  Synthetic Construct
caagcagaagacggcatacgagatctctcgtcgtctcgtgggctcgg

SEQ ID NO: 18 - DNA, 47 bp, Artificial Sequence
  Synthetic Construct
caagcagaagacggcatacgagatccaagtctgtctcgtgggctcgg

SEQ ID NO: 19 - DNA, 47 bp, Artificial Sequence
  Synthetic Construct
caagcagaagacggcatacgagatttggactcgtctcgtgggctcgg

SEQ ID NO: 20 - DNA, 47 bp, Artificial Sequence
  Synthetic Construct
caagcagaagacggcatacgagatgcagaattgtctcgtgggctcgg

SEQ ID NO: 21 - DNA, 47 bp, Artificial Sequence
  Synthetic Construct
caagcagaagacggcatacgagatatgaggccgtctcgtgggctcgg

SEQ ID NO: 22 - DNA, 47 bp, Artificial Sequence
  Synthetic Construct
caagcagaagacggcatacgagatactaagatgtctcgtgggctcgg

SEQ ID NO: 23 - DNA, 47 bp, Artificial Sequence
  Synthetic Construct
caagcagaagacggcatacgagatgtcggagcgtctcgtgggctcgg

SEQ ID NO: 24 - DNA, 47 bp, Artificial Sequence
  Synthetic Construct
caagcagaagacggcatacgagatagcctcatgtctcgtgggctcgg

SEQ ID NO: 25 - DNA, 47 bp, Artificial Sequence
  Synthetic Construct
caagcagaagacggcatacgagatgattctgcgtctcgtgggctcgg

SEQ ID NO: 26 - DNA, 47 bp, Artificial Sequence
  Synthetic Construct
caagcagaagacggcatacgagattcgtagtggtctcgtgggctcgg

SEQ ID NO: 27 - DNA, 47 bp, Artificial Sequence
  Synthetic Construct
caagcagaagacggcatacgagatctacgacagtctcgtgggctcgg

SEQ ID NO: 28 - DNA, 51 bp, Artificial Sequence
  Synthetic Construct
aatgatacggcgaccaccgagatctacacagcgctagtcgtcggcagcgtc

SEQ ID NO: 29 - DNA, 29 bp, Artificial Sequence
  Synthetic Construct
  misc_feature 29: Linked to position 1 of SEQ ID NO30 by an index sequence
aatgatacggcgaccaccgagatctacac

SEQ ID NO: 30 - DNA, 14 bp, Artificial Sequence
  Synthetic Construct
  misc_feature 1: Linked to position 29 of SEQ ID NO29 by an index sequence
tcgtcggcagcgtc